(19) United States
(12) Patent Application Publication
(10) Pub. No.: US 2020/0061087 A1
(43) Pub. Date: Feb. 27, 2020

(54) Inhibiting the Fungal Cell-Surface Phospate Transporter PHO84

(71) Applicants: Julia R. KOEHLER, Jamaica Plain, MA (US); Ningning LIU, Boston, MA (US); Children's Medical Center Corporation, Boston, MA (US)

(72) Inventors: Julia R. Koehler, Jamaica Plain, MA (US); Ningning Liu, Boston, MA (US)

(21) Appl. No.: 16/480,828

(22) PCT Filed: May 16, 2018
(86) PCT No.: PCT/US2018/032992

§ 371 Date: Jul. 25, 2019

Related U.S. Application Data
(60) Provisional application No. 62/506,897, filed on May 16, 2017.
(60) Provisional application No. 62/509,593, filed on May 22, 2017.

(51) Int. Cl. A61K 31/662; A01N 57/20; C12Q 1/18; A61P 31/10; C07K 14/395; A61K 31/122; A61K 31/7048; A61K 38/12

(52) U.S. Cl. CPC A61K 31/662; A01N 57/20; C12Q 1/18; A61P 31/10; G01N 2500/04; A61K 31/122; A61K 31/7048; A61K 38/12; C07K 14/395

(57) ABSTRACT

The invention includes a method of inhibiting Pho84 in a fungus comprising administering to the fungus an effective amount of a compound of formula 1.

### CROSS REFERENCE TO RELATED APPLICATIONS

The present application claims priority under 35 U.S.C. § 119(e) to U.S. Provisional Patent Application No. 62/506,897 filed May 16, 2017 and 62/509,593, filed May 22, 2017, each of which is incorporated herein by reference in its entirety.

### STATEMENT REGARDING FEDERALLY SPONSORED RESEARCH OR DEVELOPMENT

This invention was made with government support under Contract No. R21AI096054 and R01AI095305 awarded by the National Institutes of Allergy and Infectious Diseases. The government has certain rights in the invention.

### BACKGROUND OF THE INVENTION

Pathogenic fungi and in particular pathogenic yeast have a significant adverse impact on public health. Although antifungal drugs are available, there is a continuing need for novel compositions and methods for inhibiting fungal growth and treating infections caused by these fungi. This disclosure addresses that need.

### SUMMARY OF THE INVENTION

In one aspect, the invention provides a method of inhibiting Pho84 in a fungus comprising administering to the fungus an effective amount of a compound of formula 1:

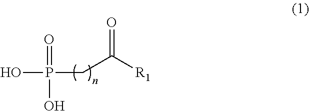

wherein n is 0 or 1 and R₁ is halogen, C₁-C₃ alkoxy, OH, H, or NH₂; or a salt or solvate thereof, thereby inhibiting Pho84 in the fungus.

In another aspect, the invention provides a method of inhibiting fungal growth comprising administering to a fungus an effective amount of a compound of formula 1:

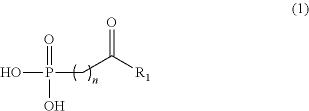

wherein n is 0 or 1 and R₁ is halogen, C₁-C₃ alkoxy, OH, H, or NH₂; or a salt or solvate thereof, thereby inhibiting fungal growth.

In yet another aspect, the invention provides a method of treating an infection caused by a fungus in a subject in need thereof comprising administering to the subject a therapeutically effective amount of a compound of formula 1:

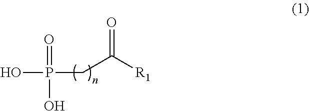

wherein n is 0 or 1 and R₁ is halogen, C₁-C₃ alkoxy, OH, H, or NH₂; or a salt or solvate thereof, thereby treating the fungal infection in the subject.

In various embodiments of the aforementioned aspects, the compound is phosphonoformic acid or phosphonoacetic acid.

In one embodiment, the therapeutically effective amount of the compound of formula 1 is administered in a pharmaceutical composition comprising at least one pharmaceutically acceptable carrier.

In various embodiments of the aforementioned aspects, wherein the fungus is from the phyla Zygomycete/Mucorales, Ascomycete, or Basidiomycete. In certain embodiments, the fungus is selected from the group consisting of: Candida parapsilosis, C. glabrata, C. lusitaniae, C. krusei, C. dublinensis, C. kefyr, C. auris, Pneumocystis jirovecii, Aspergillus fumigatus, A. flavus, A. niger, A. terreus, A. nidulans, Fusarium solani, F. oxysporum, Fusarium verticillioidis and Fusarium moniliforme, Fusarium dimerum, Fusarium proliferatum, Fusarium chlamidosporum, Fusarium sacchari, Fusarium nygamai, Fusarium napiforme, Fusarium antophilum, and Fusarium vasinfectum, Pseudoallescheria boydii, Scedosporium apiospermum, S. prolificans, Alternaria alternata, Acremonium kiliense, Blastomyces dermatitidis, Histoplasma capsulatum, Coccidioides immitis, C. posadasii, Paracoccidioides brasiliensis, Trichophyton rubrum, T tonsurans, T schoenleinii, T. verrucosum, T. mentagrophytes, T. interdigitale, T. violaceum, Microsporum canis, M. audouinii, M. ferrugineum, M. cookie, Nannizzia fulva, N. gypsea, N. persicolor, N. nana, Arthroderma insingulare, A. uncinatum, Epidermophyton floccosum, Cryptococcus gattii, Cryptococcus neoformans, Trichosporon asahii, Mucor mucedo, M. circinelloides, Rhizopus oryzae, Cunninghamella bertholletiae, Lichtheimia ramose, Rhizomucor pusillus, and Saksenaea vasiformis. In other embodiments, the fungus is a yeast. In certain embodiments, the yeast is from the genera Candida or Cryptococcus. In other embodiments, the yeast is Candida albicans or Saccharomyces cerevisiae.

In various embodiments of the aforementioned aspects of the invention, the method further comprises administering at least one additional antifungal agent. In certain embodiments, the at least one additional antifungal agent is a polyene antifungal agent, an azole antifungal agent, an allylamine antifungal agent, or an echinocandin antifungal agent. In other embodiments, the additional antifungal agent is plumbagin, amphotericin B or micafungin.

In another aspect, the invention provides a recombinant fungal cell comprising a polynucleotide encoding a reporter gene operably linked to a Pho84 promoter, wherein the fungal cell is hemizygous for Pho84.

In another aspect, the invention provides an isolated polynucleotide encoding a reporter gene operably linked to a Pho84 promoter. In one embodiment, the isolated polynucleotide comprises the sequence of SEQ ID NO: 1.

In still another embodiment, the invention provides an expression vector comprising the polynucleotide of SEQ ID NO: 1 positioned for expression in a cell.

In another aspect, the invention provides a cell comprising the aforementioned expression vector. In one embodiment, the cell is a fungal cell that is hemizygous for Pho84. In another embodiment, the fungal cell is a recombinant fungal cell comprising the isolated polynucleotide having SEQ ID NO: 1. In one embodiment, the fungal cell comprises a polynucleotide having at least 90% or 95% sequence identity to SEQ ID NO: 1. In another embodiment, the polynucleotide has a least 95% sequence identity to SEQ ID NO: 1.

In another aspect, the invention provides a method of identifying a compound that inhibits Pho84 comprising: exposing a recombinant fungal cell comprising a polynucleotide encoding a reporter gene operably linked to a Pho84 promoter to a putative inhibitor compound, wherein the fungal cell is hemizygous for Pho84; measuring the expression level of the reporter gene; and comparing the expression level of the reporter gene to a predetermined reference level; thereby identifying the compound as a Pho84 inhibitor. In various embodiments, the reporter gene is green fluorescent protein, red fluorescent protein, β-galactosidase or chloramphenicol acetyltransferase. In certain embodiments, when the level of the reporter gene differs from the predetermined reference level, it indicates that the compound is a Pho84 inhibitor.

In another aspect, the invention provides a composition comprising at least one Pho84+/− fungus cell and cell culture medium. In various embodiments, the composition further comprises at least one Pho84+/+ fungus cell.

In various embodiments, the composition further comprises a putative Pho84 inhibitor.

In certain embodiments, the cell culture medium comprises low phosphate media.

In another aspect, the invention provides a method of identifying a compound that inhibits Pho84 comprising exposing at least one Pho84+/+ fungal cell and at least one Pho84+/− fungal cell to a putative inhibitor compound; and comparing a growth level of the at least one Pho84+/+ fungal cell to a growth level of the at least one Pho84+/− fungal cell, thereby identifying the compound as a Pho84 inhibitor. In certain embodiments, the at least one Pho84+/+ fungal cell and the at least one Pho84+/− fungal cell are grown on the same media. In certain other embodiments, the growth level is determined by measuring colony size, OD₆₀₀ or the reduction of 2,3-bis-(2-methoxy-4-nitro-5-sulfophenyl)-2H-tetrazolium-5-carboxanilide (XTT) or resazurin.

In various embodiments of the aforementioned method aspects and embodiments of the invention, the subject is a mammal. In certain embodiments. the mammal is a human.

In another aspect, the invention provides a kit for identifying compounds that inhibit Pho84, comprising one or more recombinant fungal cells comprising a polynucleotide encoding a reporter gene operably linked to a Pho84 promoter, wherein the fungal cell is hemizygous for Pho84; a reagent for measuring the expression level of the reported gene; and written instructions comprising: exposing the recombinant fungal cell to a putative inhibitor compound; measuring the expression level of the reporter gene; and comparing the expression level of the reporter gene to a predetermined reference level; thereby identifying the compound as a Pho84 inhibitor.

In another aspect, the invention provides a kit for identifying compounds that inhibit Pho84, comprising one or more Pho84+/+ fungal cells and one or more Pho84+/− fungal cells; a reagent for measuring the growth level of the fungal cells; and written instructions comprising: exposing at least one Pho84+/+ fungal cell and at least one Pho84+/− fungal cell to a putative inhibitor compound; and comparing a growth level of Pho84+/+ fungal cell to a growth level of the Pho84+/− fungal cell, thereby identifying the compound as a Pho84 inhibitor.

### BRIEF DESCRIPTION OF THE DRAWINGS

For a fuller understanding of the nature and desired objects of the present invention, reference is made to the following detailed description taken in conjunction with the accompanying figures.

FIGS. 1A-1E depict data showing that C. albicans PHO84 is required for rapamycin tolerance, growth during phosphate (Pi) starvation, normal TORC1 activity and hyphal morphogenesis, and Pi homeostasis. FIG. 1A depicts cell dilutions of wild type (wt) and a mutant series in PHO84 pinned onto Yeast extract Peptone Dextrose (YPD) medium with vehicle or 12 ng/ml rapamycin. PHO84+/+, JKC915. pho84−/+, JKC1583. pho84−/−, JKC1450. pho84−/−/+, JKC1588. FIG. 1B depicts cells as in A pinned onto Yeast Nitrogen Base (YNB) medium with 0 or 11 mM Pi. FIG. 1C depicts separate Western blots of the same samples for phosphorylated ribosomal protein S6 (Rps6, P-S6), total Rps6 and tubulin of wild type (+/+, JKC915), pho84 null (−/−, JKC1450), and PHO84 reintegrant (−/−/+, JKC1588) cells grown in YNB with 0, 0.22 mM or 11 mM KH₂PO₄ for 90 min. Dens: densitometric ratio of P-S6 vs. tubulin signal. FIG. 1D depicts strains as in FIG. 1A spotted at equidistant points around agar plates and spot edges imaged. Compare with FIG. 7A. Bar 1 mm. FIG. 1E depicts S. cerevisiae and C. albicans wild type and pho84 null cells grown in Synthetic Complete (SC) medium with 0.22 mM (low Pi) or 11 mM Pi (high Pi) overnight, assayed for free and total Pi. pho85 null cells as controls which hyperaccumulate Pi. Sc+ (S. cerevisiae PHO84 PHO85), BY4741. Scpho84−, EY2960 and Scpho85−, from (37). Ca+/+ (C. albicans PHO84/PHO84 PHO85/PHO85), JKC915. Capho84−/−, JKC1450. Capho85−/−, CaLC1919, grown in 20 μg/ml doxycycline overnight. *p<0.01; **p<0.05. Error bars standard deviations (SD) of 3 technical replicates.

FIGS. 2A-2D depict data showing that Gtr1 links Pho84 to TOR in C. albicans. FIG. 2A depicts a Western blot of wt (SC5314), rhb1−/− (CCT-D1) and rhb1−/−/pADH1-RHB1 (CCT-OE1) cells, grown in YNB with 0 or 0.22 mM KH₂PO₄ for 90 min. FIG. 2B depicts growth in YPD with 4 ng/ml rapamycin or vehicle. OD₆₀₀ monitored every 15 minutes. Yellow: wt with vector (JKC1594); blue: wt overexpressing GTR1 (JKC1596); cyan: pho84−/− with vector (JKC1598); green: pho84−/− overexpressing GTR1 (JKC1600). FIG. 2C depicts cell dilutions pinned onto YPD with vehicle or 12 ng/ml rapamycin. Strains, (1) wt with vector (JKC1594), (2) wt overexpressing GTR1 (JKC1596), (3) wt overexpressing GTR1-GTP (JKC1619), (4) pho84−/− with vector (JKC1598), (5) pho84−/− overexpressing GTR1 (JKC1600) and (6) pho84−/− overexpressing GTR1-GTP (JKC1616). FIG. 2D depicts a Western blot of cells grown in YNB with 0.22 mM KH₂PO₄ for 90 min.

FIG. 3 depicts a Western blot of Saccharomyces cerevisiae (1) PHO84 with vector (Y1597), (2) pho84 with vector (Y1599), (3) PHO84 with GTR1-GTP GTR2-GDP (Y1596) and (4) pho84 with GTR1-GTP GTR2-GDP (Y1604) cells, grown in SC (-His-Ura) containing 0.22 mM and 11 mM KH₂PO₄ for 90 min, probed for P-S6, S6 and tubulin.

FIGS. 4A-4D depict data showing that small-molecule inhibition of Pho84 represses TORC1 and hyphal morphogenesis, and potentiates amphotericin B and micafungin antifungal activity. FIG. 4A depicts a Western blot of wild type (wt) (SC5314) cells with (1) vehicle, (2) 100 μM foscarnet (Fos), (3) 200 μM Fos, (4) 400 μM Fos, and strains (5, 7, 9, 11) PHO84+/+(JKC915), (6, 8) pho84−/+ (JKC1583) and (10, 12) pho84−/− (JKC1450), grown in standard SC (7.3 mM Pi) for 60 min, probed for P-S6 and tubulin. FIG. 4B depicts Wt (SC5314) spotted at equidistant points around RPMI agar (0.22 mM KH₂PO₄, pH 7) containing vehicle, 8 mM phosphonoacetic acid (PAA), or 500 μM Fos, grown at 37° C., and spot edges imaged. Compare with FIG. 7A. Bar 1 mm. FIG. 4C depicts Wt (SC5314) exposed to vehicle, 500 μM Fos, 0.2 μg/ml amphotericin B, and amphotericin B plus Fos; OD₆₀₀ in SC with 0.5 mM KH₂PO₄ at 30° C. monitored every 15 minutes. FIG. 4D depicts Wt (SC5314) exposed to vehicle, 500 μM Fos, 25 ng/ml micafungin, and micafungin plus Fos; OD₆₀₀ in SC with 0.5 mM KH₂PO₄ at 30° C. monitored every 15 minutes.

FIG. 5 is a schematic depicting the interaction between the various proteins involved in the system. Pho84 activates TORC1 via Gtr1, and TORC1 in turn modulates the PHO regulon. Foscarnet inhibits Pho84 and thereby indirectly blocks TORC1 activity. Signaling events with known molecular mechanisms in S. cerevisiae are shown as green lines. Blue lines represent predicted activities.

FIGS. 6A-6F depict data showing that C. albicans PHO84 is required for normal TORC1 activity and is an ortholog of S. cerevisiae PHO84 and a component of the C. albicans PHO regulon. FIG. 6A depicts cell dilutions of wild type (wt) SC5314 and its derivative pho84::Tn mariner::NAT1/PHO84 pinned onto YPD with vehicle (90% ethanol) or 12 ng/ml rapamycin. FIG. 6B depicts a sequence alignment showing that amino acid identities and similarities between C. albicans and S. cerevisiae Pho84 orthologs are 66% and 78%, respectively. Amino acid identities between C. albicans Pho84 and the P. indica PiPT whose crystal structure was recently elucidated are 38% and similarities are 55%. FIG. 6C is an image showing dilutions of cell suspensions of C. albicans lineages derived from 2 independently generated heterozygous pho84/PHO84 deletion mutants pinned onto YPD medium with 90% ethanol, synthetic complete (SC) medium devoid of Pi, and YPD with 8 ng/ml rapamycin. 1. CaPHO84+/+ wild type (JKC915). 2. Capho84−/+ heterozygote (JKC1580). 3. Capho84−/+ heterozygote (JKC1583). 4. Capho84−/− homozygote (JKC1430). 5. Capho84−/− homozygote (JKC1450). 6. Capho84−/−/+ reintegrant (JKC1586). 7. Capho84 reintegrant−/−/+ (JKC1588). FIG. 6D depicts growth curves in SC medium with 0, 1 mM and 5 mM KH₂PO₄. OD₆₀₀ monitored every 15 minutes. Blue: wt+/+(JKC915); green: pho84−/+ (JKC1583); cyan: pho84−/− (JKC1450); purple: pho84−/−/+(JKC1588). FIG. 6E depicts an image in which S. cerevisiae cell dilutions were pinned onto SC-his with 0 or 11 mM Pi. ScPHO84<vector>, JKY1471. Scpho84<vector>, JKY1482. Scpho84<ScPHO84>, JKY1494. Scpho84<CaPHO84>, JKY1486. FIGS. 6C and 6D are representatives of 3 biological replicates. FIG. 6F is a graph depicting data from an assay in which Cells were grown overnight in YNB with 11 mM or with 0 Pi, and secreted acid phosphatase was assayed. +/+ (PHO84/PHO84), JKC915. −/− (pho84/pho84), JKC1450. In FIGS. 6E and 6F, error bars: SD of 3 technical replicates, representing 3 biological replicates. p was calculated comparing pho84−/− null mutant with wild type using Student's t-test.

FIGS. 7A and 7B depict data showing that cells lacking C. albicans PHO84 exhibit robust growth in the same media in which their hyphal morphogenesis is defective. FIG. 7A depicts a filamentation assay: in order to maximize reproducibility between replicate experiments, cells were suspended in 0.9% NaCl to an OD₆₀₀ of 0.1; then 3 μl of the suspension was spotted at equidistant points, using a template, around an RPMI 1640 agar plate buffered to pH7 with 50 mM MOPS containing 0.22 mM KH₂PO₄, as in FIG. 4B. Spot edges were imaged to show the length and density of filaments extending from the edge of the spot into the agar medium. Scale bar 1 mm. PHO84+/+, JKC915. pho84+/−, JKC1583. pho84−/−, JKC1450. pho84-/−/+, JKC1588. FIG. 7B depicts growth curves at 30° C. in YPD medium with 10% serum, Spider medium and RPMI medium with 0.22 mM KH₂PO₄ buffered to pH7 with 50 mM MOPS. OD₆₀₀ monitored every 15 minutes. Blue: wt+/+ (JKC915); green: pho84−/+ (JKC1583); cyan: pho84−/− (JKC1450); purple: pho84−/−/+ (JKC1588). Error bars represent SD of 3 technical replicates. Panels A, B: Representatives of 3 biological replicates are shown.

FIGS. 8A-8C depict data showing that the connection between Pho84 and TORC1 is divergent between C. albicans and S. cerevisiae, but as in C. albicans, S. cerevisiae TORC1 activity can be assayed by P-S6 signal intensity. FIG. 8A depicts an assay in which S. cerevisiae cell suspensions were pinned onto synthetic complete medium lacking histidine and uracil (SC) with 90% ethanol, SC devoid of Pi, and SC with 20 ng/ml rapamycin. 1. PHO84 with vectors (JKY1597). 2. PHO84 with GTR1-GTP GTR2-GDP (JKY1596). 3. pho84 with vectors (JKY1599). 4. pho84 with ScPHO84 (JKY1601). 5. pho84 with GTR1-GTP GTR2-GDP (JKY1604). A representative of 3 biological replicates is shown. FIG. 8B depicts a Western blot of S. cerevisiae cells 1. Wild type (wt) PHO84 with vector (Y1597), 2. pho84 with vector (Y1599), 3. PHO84 with GTR1-GTP GTR2-GDP (Y1596) and 4. pho84 with GTR1-GTP GTR2-GDP (Y1604) cells, grown in SC (-His-Ura) containing 0.22 mM and 11 mM KH2PO4 for 90 min, probed for P-Sch9, Sch9, and PSTAIRE. FIG. 8C depicts a Western blot for P-S6 of S. cerevisiae wild type with vectors (JKY1652), grown in YNB for 90 minutes. Low (1 mM) and and high (10 mM) concentrations of non-preferred (proline), moderately preferred (leucine) and preferred (glutamine) nitrogen sources (2) were provided to compare the P-S6 signal as a readout of TORC1 activation. Time 0, overnight culture (1). (2) YNB medium containing 10 mM methionine with addition of 1 mM proline plus vehicle (90% ethanol), (3) 10 mM proline plus vehicle, (4) 1 mM glutamine plus vehicle, (5) 10 mM glutamine plus vehicle, (6) 1 mM leucine plus vehicle, (7) 10 mM leucine plus vehicle, (8) 10 mM glutamine plus 100 nM rapamycin, and (9) 10 mM glutamine plus vehicle, treated with 800 U λ phosphatase. The same lysate samples were separately electrophoresed to probe for P-S6 or total S6. Tubulin was the loading control for both blots. A representative of 3 biological replicates is shown.

FIGS. 9A and 9B depict data showing that TORC1 modulates the PHO regulon in C. albicans. FIG. 9A depicts an assay in which PHO84 mRNA levels were measured by RT-PCR in strains TOR1/TOR1 (JKC1361) (1), tor1/tetO-TOR1Δ1-381 (JKC1441) (2) and tor1/tetO-TOR1 (JKC1549) (3). Cells were diluted from overnight culture (TO) into fresh YPD medium with 30 μg/ml doxycycline repressing expression from tetO. The ratio of PHO84/ACT1 mRNA at 2 and 4 hours relative to the overnight culture (TO) was graphed. tor1/tetO-TOR1Δ1-381 in comparison with TOR1/TOR1 at 2 h p=0.0102 and at 4 h p<0.0001. tor1/tetO-TOR1 in comparison with TOR1/TOR1 at 2 h p=0.0306 and at 4 h p<0.0001. FIG. 9B depicts an assay in which wells were grown overnight in YNB medium with 11 mM Pi, and secreted acid phosphatase was assayed. Strains are (1) PHO84/PHO84 (+/+) with vector (JKC1636), (2) +/+ overexpressing GTR1 (JKC1627) (in comparison with +/+ with vector, p=0.1646), (3) +/+ overexpressing GTR1-GTP (JKC1630) (in comparison with +/+ with vector, p=0.8135), (4) pho84/pho84 null (−/−) with vector (JKC1598), (in comparison with +/+ with vector, p<0.0001), (5) −/− overexpressing GTR1 (JKC1600) (in comparison with −/− with vector, p<0.0001) and (6) −/− overexpressing GTR1-GTP (JKC1616) (in comparison with −/− with vector, p<0.0001). FIGS. 9A and 9B are representative of 3 biological replicates; error bars show SD. p values were calculated using Student's t-test.

FIGS. 10A-10D depict data showing that Foscarnet inhibits Pho84 in competition with Pi. Wild type PHO84/PHO84 (+/+, dark blue) (JKC915) and pho84/pho84 null (−/−, light blue) (JKC1450) cells were exposed to 1 and 4 mM PAA (FIG. 10A) or 200 and 400 μM foscarnet (FIG. 10B), and growth in SC medium with 0.22 mM or 11 mM KH2PO4 at 30° C. was monitored automatically every 15 minutes. FIGS. 10A and 10B are representative of 3 biological replicates; error bars show SD. FIG. 10C depicts a filamentation assay: in order to maximize reproducibility between replicate experiments, cells of wild type strain SC5314 were suspended in 0.9% NaCl to an OD600 of 0.1; then 3 μl of the suspension was spotted at equidistant points, using a template, around a Spider agar plate containing vehicle or 500 μM foscarnet. Spot edges were imaged to show the length and density of filaments extending from the edge of the spot into the agar medium. Scale bar 1 mm. A representative of 3 biological replicates is shown. FIG. 10D depicts data from an assay in which wild type SC5314 was exposed to vehicle, 8 mM PAA, 0.25 μg/ml amphotericin B, and amphotericin B plus PAA, and grown in SC medium with 0.5 mM KH2PO4 at 30° C. OD600 was monitored automatically every 15 minutes. Error bars are SD of 3 technical replicates. A representative of 3 biological replicates is shown.

FIGS. 11A-11D depict data showing that virulence is attenuated in C. albicans cells lacking PHO84. FIG. 11A shows the percentage of surviving Drosophila melanogaster after five days' infection with +/+ (PHO84/PHO84), JKC915; −/+ (pho84/PHO84), JKC1583; −/− (pho84/pho84), JKC1450 and 4-1+ (pho84/pho84::PHO84), JKC1588 respectively. The flies were injected with 50 nl fungal cell suspensions with a micro-injector. At least 3 (up to 7) biological replicates (independently prepared fungal preparations derived from individual colonies) and 6-15 technical replicates per strain were carried out. FIG. 11B depicts the oral fungal burden of mice with oropharyngeal candidiasis was calculated by enumerating CFU per gram tongue tissue after 5 days of infection. FIG. 11C depicts a Kaplan-Meier survival plot of mice with disseminated candidiasis. Eight 6-to-8-week-old female BALB/c mice were injected with +/+ (PHO84/PHO84), JKC915; −/− (pho84/pho84), JKC1450 and −/−/+(pho84/pho84::PHO84), JKC1588 respectively. FIG. 11D depicts kidneys from +/+ (PHO84/PHO84), JKC915; −/− (pho84/pho84), JKC1450 and −/−/+ (pho84/pho84::PHO84), JKC1588 infected mice were isolated and stained with Grocott-Gomori methenamine-silver (GMS).

FIGS. 12A-12E depict data showing Pho84 is required for resistance to killing by whole blood or neutrophils, in dependence on neutrophil reactive oxygen species (ROS). FIG. 12A depicts percent survival of C. albicans cells after incubation with heparinized whole blood from healthy human volunteers; +/+ (PHO84/PHO84), JKC915; −/− (pho84/pho84), JKC1450 and −/−/+(pho84/pho84::PHO84), JKC1588 were inoculated at 5×103/ml into blood which was plated onto agar medium at the indicated time points, and CFU/ml were calculated. pho84/pho84 in comparison with PHO84/PHO84 at 5 h p=0.008. pho84/pho84::PHO84 in comparison with PHO84/PHO84 at 5 h p=0.07. FIG. 12B depicts percent survival of C. albicans cells after incubation of HL-60 derived neutrophils with +/+ (PHO84/PHO84), JKC915; −/− (pho84/pho84), JKC1450 (p=0.006 in comparison with PHO84/PHO84) and −/−/+ (pho84/pho84::PHO84), JKC1500 (p=0.14 in comparison with PHO84/PHO84) at a 20:1 phagocyte: fungus ratio. FIG. 12C depicts percent survival of C. albicans cells after incubation with human neutrophils, +/+ (PHO84/PHO84), JKC915; −/− (pho84/pho84), JKC1450 and −/−/+ (pho84/pho84::PHO84), JKC1588 at M.O.I. 2 for 2 hrs and 5 hrs. pho84/pho84 in comparison with PHO84/PHO84 at 5 h p=0.04. pho84/pho84::PHO84 in comparison with PHO84/PHO84 at 5 h p=0.69. FIG. 12D depicts human peripheral blood derived neutrophils pretreated with different concentrations of N-acetyl-1-cysteine (NAC) were incubated for 90 min with +/+ (PHO84/PHO84), JKC915; −/− (pho84/pho84), JKC1450 and −/−/+ (pho84/pho84::PHO84), JKC1588 at M.O.I. 2. pho84/pho84 in comparison with PHO84/PHO84 with vehicle p<0.0001. pho84/pho84 in comparison with PHO84/PHO84 with 10 mM N-acetyl glucosamine (NAC) p=0.1258. FIG. 12E depicts human peripheral blood derived neutrophils pretreated with 10 μM Diphenyleneiodonium (DPI) were incubated for 90 min with +/+ (PHO84/PHO84), JKC915; −/− (pho84/pho84), JKC1450 and −/−/+(pho84/pho84::PHO84), JKC1588 at M.O.I. 2. Vehicle alone, DPI alone and neutrophil alone groups are controls. p=0.3 for pho84/pho84 in neutrophils+DPI versus PHO84/PHO84 in neutrophils+DPI. p=0.0.87 for pho84/pho84::PHO84 in neutrophils+DPI versus PHO84/PHO84 in neutrophils+DPI. p values were calculated using Student's t-test. FIGS. 12A-12D show representatives of at least 3 biological replicates.

FIGS. 13A-13C depict data showing that Pho84 is required for virulence in cell damage assays. FIG. 13A depicts data from a damage assay with the FaDu epithelial cell line. FIG. 13B depicts data from a damage assay with human umbilical vein endothelial cells (HUVEC). FIG. 13C depicts data from a neutrophil killing assay.

FIGS. 14A and 14B are a pair of graphs depicting C. albicans resistance to platelet-dependent inhibition in untreated, thrombocytopenic and reconstituted blood for wild type (FIG. 14A) and Pho84 null mutant (FIG. 14B).

FIG. 15 is a graph depicting the survival rate of C. albicans Pho84 wild type, null mutant and reintegrant over time when exposed to neutrophils isolated from healthy volunteers.

FIGS. 16A and 16B depict data showing that resistance to oxidative stressors is decreased, and Hog1 phosphorylation is increased in cells lacking PHO84. In FIG. 16A, overnight cultures were washed with normal saline and diluted to YPD with vehicle, 25 μM plumbagin, 40 μM menadione, and 40 mM H₂O₂ to OD₆₀₀ 0.1. OD₆₀₀ was monitored every 15 minutes for strains +/+ (PHO84/PHO84), JKC915; −/− (pho84/pho84), JKC1450 and −/−/+(pho84/pho84::PHO84), JKC1588. FIG. 16B depicts a Western blot of cells incubated in YPD containing 5 mM H2O2 for 5 min, 10 min, 20 min, 30 min and 40 min. The membrane was probed for P-Hog1, total Hog1, and loading controls PSTAIRE and Histone H3. Strains, +/+ (PHO84/PHO84), JKC915; −/− (pho84/pho84), JKC1450 and −/−/+ (pho84/pho84::PHO84), JKC1588. FIGS. 16A and 16B show representatives of at least 3 biological replicates.

FIG. 17 depicts images (left) and a graph of fluorescence intensity (right) for wild type, null mutant and reintegrant indicating that loss of Pho84 leads to accumulation of ROS over time.

FIG. 18 depicts images (top) and graphs presenting integrated intensity (below) demonstrating that the accumulation of ROS in Pho84−/− null cells can be partially rescued by increased ambient Mn concentration.

FIGS. 19A-19F depict data indicating that reactive oxygen species (ROS) management, Sod1 activity and Sod3 expression are defective in cells lacking PHO84, but metal SOD co-factors are not diminished. FIG. 19A shows DCFDA detectable ROS of cells unexposed to extrinsic oxidative stress, strains +/+ (PHO84/PHO84), JKC915; −/− (pho84/pho84), JKC1450 and −/−/+ (pho84/pho84::PHO84), JKC1588 were diluted into SC medium with 0.22 mM, 1 mM and 11 mM Pi. Fluorescence intensity was measured after staining cells with 50 μM DCFH-DA. pho84/pho84 versus with PHO84/PHO84 at 0.22 mM Pi p=0.0149. pho84/pho84::PHO84 versus with PHO84/PHO84 at 0.22 mM Pi p=0.3782. pho84/pho84 versus with PHO84/PHO84 at 1 mM Pi p<0.0001. pho84/pho84::PHO84 versus PHO84/PHO84 at 1 mM Pi p=0.0007. pho84/pho84 versus PHO84/PHO84 at 11 mM Pi p<0.0001. pho84/pho84::PHO84 versus PHO84/PHO84 at 11 mM Pi p<0.0001. p values calculated using Student's t-test. FIG. 19B depicts DCFDA detectable ROS production during exposure to 100 μM menadione. +/+ (PHO84/PHO84), JKC915; −/− (pho84/pho84), JKC1450 and −/−/+ (pho84/pho84::PHO84), JKC1588 (p<0.0001) cells cultured overnight were diluted into SC medium (Loflo) and fluorescence intensity was measured as in A. p=0.0002 for pho84/pho84 versus PHO84/PHO84. FIG. 19C depicts Superoxide dismutase activity of +/+ (PHO84/PHO84), JKC915; −/− (pho84/pho84), JKC1450 and −/−/+ (pho84/pho84::PHO84), JKC1588 cells, grown in YPD medium with vehicle, 50 μM Menadione and 0.8 mM bathocuproine disulfonic acid (BCS) for 8 hours; cell lysate in non-denaturing gel stained with nitrotetrazolium blue to detect superoxide dismutase activity and with Coomassie blue to assess loading. FIG. 19D depicts a Western blot of +/+ (PHO84/PHO84), JKC915; −/− (pho84/pho84), JKC1450 and −/−/+ (pho84/pho84::PHO84), JKC1588 cells, grown in normal SC medium with vehicle, 3 mM MnCl2 and 3 mM CuSO4 for 13 hours, probed for Sod3 and loading control tubulin. FIG. 19E depicts Total intracellular copper of strains +/+ (PHO84/PHO84), JKC915; −/− (pho84/pho84), JKC1450 and −/−/+ (pho84/pho84::PHO84), JKC1588, grown in normal SC medium with vehicle, 3 mM MnCl2 and 3 mM CuSO4 for 13 hours was measured by Atomic Absorption Spectroscopy (AAS). Panel F depicts the total intracellular manganese of strains +/+ (PHO84/PHO84), JKC915; −/− (pho84/pho84), JKC1450 and −/−/+ (pho84/pho84::PHO84), JKC1588, grown in normal SC medium with vehicle, 3 mM MnCl2 and 3 mM CuSO4 for 13 hours was measured by AAS. FIGS. 19A-19C and FIGS. 19D-19F show representatives of at least 3 biological replicates.

FIGS. 20A-20D depict data showing that TORC1 activity contributes to ROS management and Sod3 expression. FIG. 20A depicts dichlorodihydrofluorescein diacetate (DCFDA) detectable ROS measurement of strains, (1) PHO84/PHO84 containing vector (JKC1594), (2) pho84/pho84 containing vector (JKC1598), (3) PHO84/PHO84 overexpressing GTR1 (JKC1596), (4) pho84/pho84 overexpressing GTR1 (JKC1600). Cells cultured overnight in YPD were diluted into SC medium (Loflo) with vehicle or 8 ng/ml rapamycin for 1 hour, and fluorescence intensity was determined at excitation-485 nm and emission wavelength 528 nm after staining cells with 50 μM DCFDA. p=0.0252 for ratio of pho84−/− with vector and pho84−/− overexpressing GTR1 versus ratio of wild type with vector and wild type overexpressing GTR1; all cells exposed to vehicle. p=0.0014 for ratio of pho84−/− with vector and pho84−/− overexpressing GTR1 versus ratio of wild type with vector and wild type overexpressing GTR1; all cells exposed to rapamycin. p values calculated using Student's t-test. FIG. 20B depicts a Western blot of strains, (1) PHO84/PHO84 containing vector (JKC1594), (2) PHO84/PHO84 overexpressing GTR1 (JKC1596), (3) pho84/pho84 containing vector (JKC1598), (4) pho84/pho84 overexpressing GTR1 (JKC1600), grown in SC medium (Loflo) for 1 hour, probed for Sod3 and loading control tubulin. In FIG. 20C, wild type (SC5314) cells were exposed to vehicle, 500 microM foscarnet or 1 mM PAA for 1 hour, and fluorescence intensity was determined at excitation-485 nm and emission wavelength 528 nm after staining cells with 50 μM DCFH-DA. p<0.001 for both 500 microM foscarnet versus vehicle and 1 mM PAA versus vehicle. In FIG. 20D, dilutions of cell suspensions of C. albicans strains were pinned onto YPD medium with 0 or 0.3 μg/ml doxycycline with vehicle, 5 mM H2O2 and 20 μM plumbagin. 1. TOR1/TOR1 (JKC1361). 2. tor1/TOR1 (JKC1345). 3. tor1/TOR1 (JKC1346). 4. tor1/TOR1 (JKC1347). 5. tor1/tetO-TOR1Δ1-381 (JKC1442). 6. tor1/tetO-TOR1Δ1-381 (JKC1445). 7. tor1/tetO-TOR1Δ1-381 (JKC1441). 8. tor1/tetO-TOR1 (JKC1543). 9. tor1/tetO-TOR1 (JKC1546). 10. tor1/tetO-TOR1 (JKC1549). FIGS. 20A-20D show representatives of at least 3 biological replicates.

FIG. 21 is a graph showing that a reporter strain induces GFP in response to reduced phosphate (Pi) signaling. Flow cytometry for GFP expression of a strain pho84/PHO84 (−/+) expressing a PHO84 promoter-GFP fusion, cultured in decreasing concentrations of Pi for 2 hours after overnight excess Pi feeding: A (bright red):10 mM; B (blue):1 mM; C (green):0.5 mM; D (orange):0.2 mM; E (cyan):0.1 mM; F (mauve): 0.05 mM; G (light brown): 0.02 mM; H (dark brown): 0. X axis: fluorescence intensity. Y axis: Number of events ×10³.

FIG. 22 is a sequence alignment showing the conservation of Pho84 homologs in other fungal phyla including Basidiomycota, Ascomycota and Mucorales, as well as the pathogenic amoeba Entamoeba histolytica.

FIG. 23 depicts data showing that an oxidative stressor reveals stress intensity-dependent haploinsufficiency of pho84/pho84::PHO84 reintegrant cells. Overnight cultures were washed with normal saline and diluted to YPD with vehicle, 20 μM plumbagin, 22 μM plumbagin, 25 μM plumbagin and 28 μM plumbagin to OD₆₀₀=0.1. OD₆₀₀ was monitored every 15 minutes. Strains used were: +/+ (PHO84/PHO84), JKC915; −/− (pho84/pho84), JKC1450 and −/−/+(pho84/pho84::PHO84), JKC1588.

FIGS. 24A-24C depicts data showing that Pho84 does not affect induction of phagocytosis or ROS production by neutrophils. In FIG. 24A, +/+ (PHO84/PHO84 promoterACT1-GFP), JKC1648; −/− (pho84/pho84 promoterACT1-GFP), JKC1651 and −/−/+(pho84/pho84::PHO84 promoterACT1-GFP), JKC1653 cells were incubated with neutrophils at the M.O.I. 2 and M.O.I. 10. Phagocytosing neutrophils were quantified as CD11b+GFP+ Cells. In FIG. 24B, intracellular ROS production by neutrophils was measured after stimulation with C. albicans yeast at the M.O.I. 2 for 30 minutes. In FIG. 24C, extracellular ROS production was measured by incubation with C. albicans yeast at M.O.I. 2 for 1 hour in the presence of 100 mM Cytochrome C. Strains used were: +/+ (PHO84/PHO84), JKC915; −/− (pho84/pho84), JKC1450 and −/−/+ (pho84/pho84::PHO84), JKC1588.

FIGS. 25A and 25B show how overexpression of SOD3 from a heterologous promoter significantly rescues ROS hypersensitivity of cells lacking Pho84. Cells were grown on YPD agar medium without or with 50 ng/ml doxycycline for 39 hrs, and were maintained in these doxycycline concentrations throughout the course of the experiment. Cells were inoculated at OD600 0.1 in YPD (glucose-containing medium that represses transcription from pMAL2), with vehicle DMSO (V) or 21 μM Plumbagin (P). OD600 was monitored every 15 minutes for strains (FIG. 25A) wild type background: +/+, JKC915; +/+ tetO-SOD3/SOD3, JKC1738; +/+ pMAL2-SOD3/SOD3, 1001 JKC1776; (FIG. 25B) pho84 null mutant background: −/−, JKC1450; tetO-SOD3/SOD3, JKC1745; −/− 1002 pMAL2-SOD3/SOD3, JKC1780. FIGS. 25A and 25B are representative of 3 biological replicates, error bars SD of 3 technical replicates.

### DEFINITIONS

The instant invention is most clearly understood with reference to the following definitions.

As used herein, the singular form “a,” “an,” and “the” include plural references unless the context clearly dictates otherwise.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Certain specific examples include (C₁-C₃)alkyl, such as, but not limited to, ethyl, methyl, propyl, and isopropyl, isobutyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Certain specific examples include (C₃-C₆)cycloalkyl, such as, but not limited to, cyclopropyl, cyclobutyl, cyclopentyl and cyclohexyl.

The term “alkoxy,” as used herein, refers to an alkyl or a cycloalkyl group which is linked to another moiety though an oxygen atom.

The terms “biomarker” or “marker,” as used herein, refers to a molecule that can be detected. Therefore, a biomarker according to the present invention includes, but is not limited to, a nucleic acid, a polypeptide, a carbohydrate, a lipid, an inorganic molecule, an organic molecule, each of which may vary widely in size and properties. A “biomarker” can be a bodily substance relating to a bodily condition or disease or it may be the product of an exogenous gene, such as but not limited to a reporter gene, expressed in vitro or in vivo, in cells, tissue or a cell free expression system. A “biomarker” can be detected using any means known in the art or by a previously unknown means that only becomes apparent upon consideration of the marker by the skilled artisan.

A “compound” as used herein, may be a small molecule, a nucleic acid or a peptide.

An “effective amount” or “therapeutically effective amount” of a compound is that amount of compound which is sufficient to provide the intended effect when the compound is administered. An “effective amount” of a delivery vehicle is that amount sufficient to effectively bind or deliver a compound.

The term “halogen” or “halo” designates —F, —Cl, —Br or —I.

The term “differ”, as used herein and applied to the relative amount of a level of a biomarker with respect to a predetermined reference level, means that the levels are not the same, that the level of the biomarker is greater than or less than the predetermined reference level.

The term “inhibitor”, as used herein refers to a compound that specifically binds and reduces the biological activity of a target. The inhibitor may bind the target at the level of protein, by way of non-limiting example, by binding to the active site and excluding the natural substrate. The inhibitor may also bind the target at the level of nucleic acid, by way of non-limiting example, siRNA techniques.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The “level” of one or more biomarkers means the absolute or relative amount or concentration of the biomarker in the sample as determined by measuring mRNA, cDNA, small organic molecules, nucleotides, ions or protein, or any portion thereof such as oligonucleotide or peptide. A level of a biomarker may refer, based on context, to a global level or a level within some subdivision of an organism, by way of non-limiting example a level may refer to the amount or concentration of a biomarker in a cell, in a particular type of cell, on the cell membrane, in an area delineated by another marker or any other configuration.

The terms “patient,” “subject,” “individual,” and the like are used interchangeably herein, and refer to any animal, mammalian or non-mammalian, human or non-human, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids or bases, organic acids or bases, solvates, hydrates, or clathrates thereof.

A “reference level” of a biomarker means a level of a biomarker that is indicative of the presence or absence of a particular phenotype or characteristic, including but not limited to fluorescence by a reporter gene, cellular growth, etc. When the level of a biomarker in a subject is above the reference level of the biomarker it is indicative of the presence of, or relatively heightened level of, a particular phenotype or characteristic. When the level of a biomarker in a subject is below the reference level of the biomarker it is indicative of a lack of or relative lack of a particular phenotype or characteristic.

As used herein, a “reporter gene construct” refers to a nucleic acid that includes a “reporter gene” operatively linked to transcriptional regulatory sequences. Transcription of the reporter gene is controlled by these sequences. The activity of at least one or more of these control sequences is directly or indirectly regulated by a target protein. The transcriptional regulatory sequences include the promoter and other regulatory regions, such as enhancer sequences, that modulate the activity of the promoter, or regulatory sequences that modulate the activity or efficiency of the RNA polymerase that recognizes the promoter, or regulatory sequences which are recognized by effector molecules, including those that are specifically induced by interaction of an extracellular signal with the target receptor. For example, modulation of the activity of the promoter may be affected by altering the RNA polymerase binding to the promoter region, or, alternatively, by interfering with initiation of transcription or elongation of the mRNA. Such sequences are herein collectively referred to as transcriptional regulatory elements or sequences. In addition, the construct may include sequences of nucleotides that alter translation of the resulting mRNA, or stability of the resulting translated protein, thereby altering the amount of reporter gene product. The reporter gene constructs of the present invention provide a detectable readout, e.g., fluorescences, in response to signals transduced in response to modulation of a target by an inhibitor.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

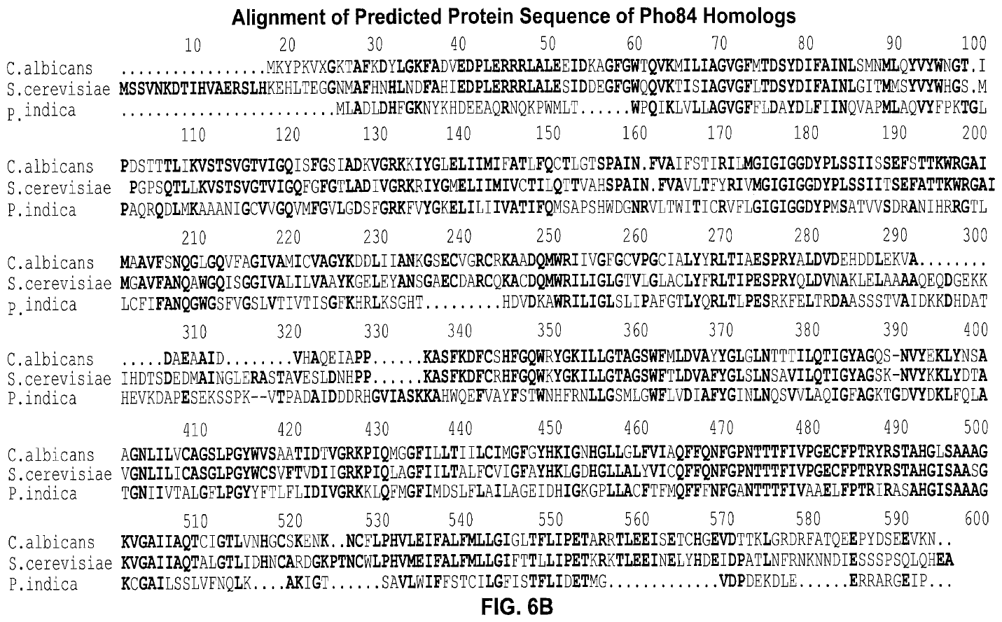

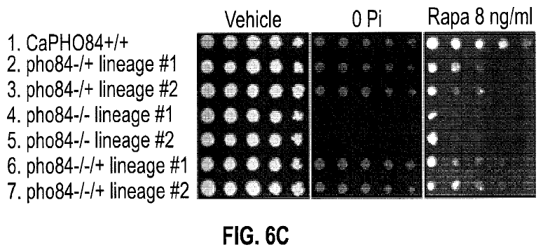

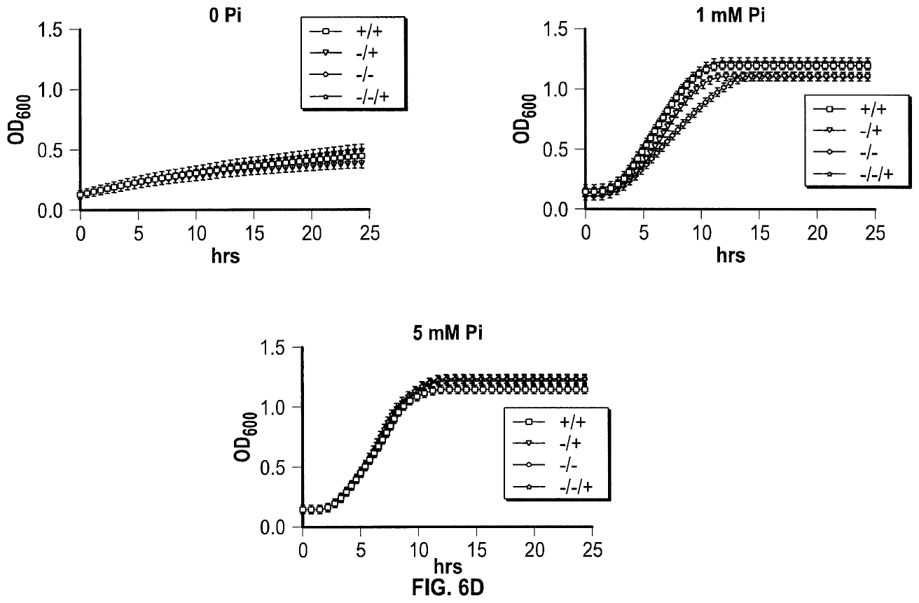

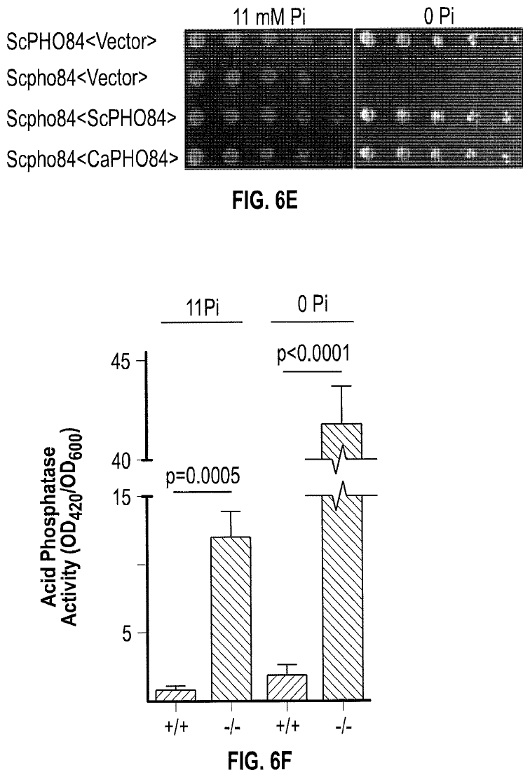

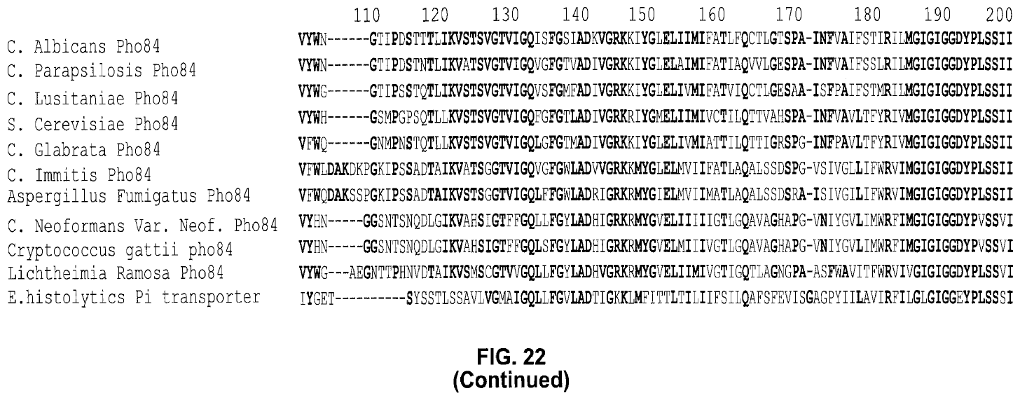

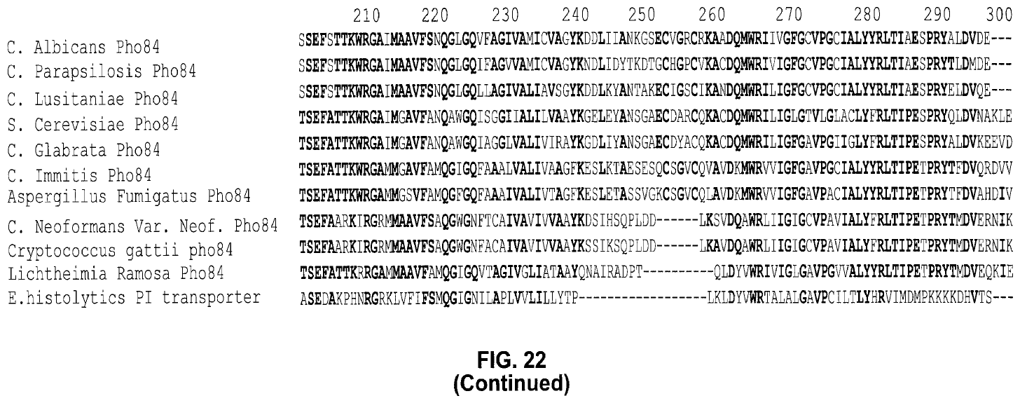

The invention is based, at least in part, on the discovery that compounds of formula (1) inhibit Pho84 which is important for the growth, survival and virulence of various strains of fungus. Without wishing to be bound by theory, Pho84 is highly conserved across various classifications of fungus. FIGS. 6 and 22 show sequence alignments of Pho84 homologs from various species, these data are discussed in Example 17. The high degree of sequence similarity and identity among these homologs implies that the compound of formula (1) will be effective against various types of fungus. Unexpectedly, Pho84 is also conserved in Entamoeba histolytica and in various further aspects the methods may be applied to this species and amoeba generally.

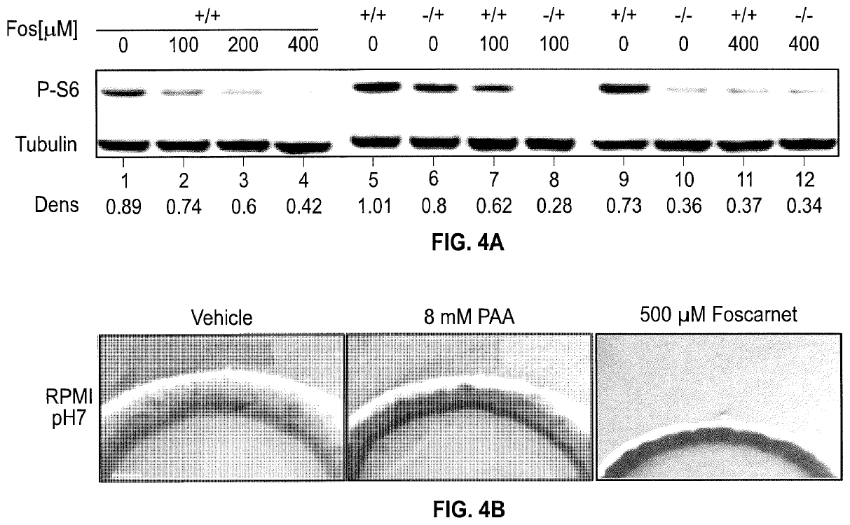

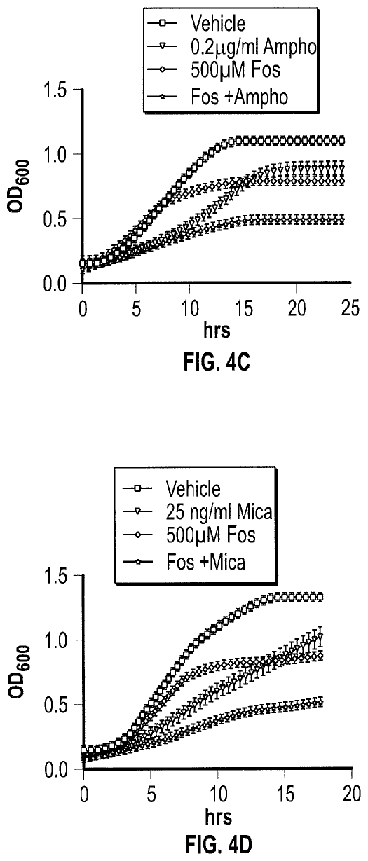

In specific embodiments, the compound of formula (1) can be phosphonoacetic or phosphonoformic acid. Data is presented in FIGS. 4, 5 and 10 and discussed in Example 7.

### Methods of Inhibiting Pho84 in a Fungal Cell

In one aspect the invention provides a method of inhibiting Pho84 in a fungus comprising administering to the fungus an effective amount of a compound of formula 1:

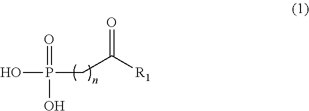

wherein n is 0 or 1 and R₁ is halogen, C₁-C₃ alkoxy, OH, H, or NH₂;

or a salt or solvate thereof, thereby inhibiting Pho84 in the fungus.

In another aspect, the invention provides a method of inhibiting fungal growth comprising administering to a fungus an effective amount of a compound of formula 1:

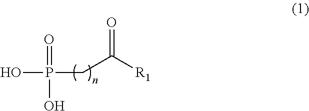

wherein n is 0 or 1 and R₁ is halogen, C₁-C₃ alkoxy, OH, H, or NH₂;

or a salt or solvate thereof, thereby inhibiting fungal growth.

In another aspect, the invention provides a method of treating an infection caused by a fungus comprising administering to a fungus an effective amount of a compound of formula 1:

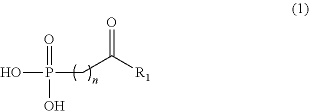

wherein n is 0 or 1 and R₁ is halogen, C₁-C₃ alkoxy, OH, H, or NH₂;

or a salt or solvate thereof, thereby treating the infection.

The following embodiments apply to one or more of the above described aspects.

In various embodiments, the compound of formula (1) may be provided in a pharmaceutical composition including a pharmaceutically acceptable carrier. Any route of administration may be employed as deemed appropriate by persons of skill in the art. Accordingly, any form of pharmaceutical composition may be employed and may include any appropriate pharmaceutical carrier or excipient. Specific examples that may be used are described below.

The methods of the invention may be applied to any fungus, including yeasts. In various embodiments the fungus may be from the phyla Zygomycete/Mucorales, Ascomycete, or Basidiomycete. In various embodiments the fungus is Candida parapsilosis, C. glabrata, C. lusitaniae, C. krusei, C. dublinensis, C. kefyr, C. auris, Pneumocystis jirovecii, Aspergillus fumigatus, A. flavus, A. niger, A. terreus, A. nidulans, Fusarium solani, F. oxysporum, Fusarium verticillioidis and Fusarium moniliforme, Fusarium dimerum, Fusarium proliferatum, Fusarium chlamidosporum, Fusarium sacchari, Fusarium nygamai, Fusarium napiforme, Fusarium antophilum, and Fusarium vasinfectum Pseudoallescheria boydii, Scedosporium apiospermum, S. prolificans, Alternaria alternata, Acremonium kiliense, Blastomyces dermatitidis, Histoplasma capsulatum, Coccidioides immitis, C. posadasii, Paracoccidioides brasiliensis, Trichophyton rubrum, T. tonsurans, T. schoenleinii, T. verrucosum, T. mentagrophytes, T. interdigitale, T. violaceum, Microsporum canis, M. audouinii, M. ferrugineum, M. cookie, Nannizzia fulva, N. gypsea, N. persicolor, N. nana Arthroderma insingulare, A. uncinatum, Epidermophyton floccosum, Cryptococcus gattii, Cryptococcus neoformans, Trichosporon asahii, Mucor mucedo, M circinelloides, Rhizopus oryzae, Cunninghamella bertholletiae, Lichtheimia ramose, Rhizomucor pusillus or Saksenaea vasiformis. In various embodiments, the fungus may be a yeast and the yeast may be selected from Candida or Cryptococcus. In various embodiments the yeast may be C. albicans or S. cerevisiae.

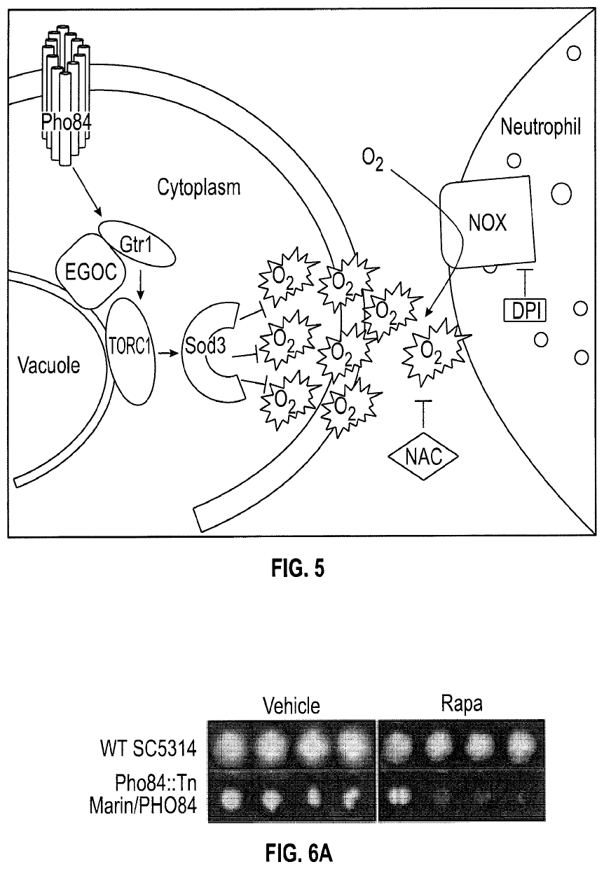

In various embodiments, the compound of formula (1) may be administered in combination with at least one additional antifungal agent. Without wishing to be limited by theory, inhibition of Pho84 sensitizes fungal cells against various other antifungal compounds. Data supporting this are presented in FIGS. 4 and 5 and Example 7. The additional antifungal agents may be any known or heretofore unknown antifungal compound suitable for the intended purpose as recognized by a person of skill in the art. In various embodiments, the antifungal compound may be a polyene antifungal agent, an azole antifungal agent, an allylamine antifungal agent, or an echinocandin antifungal agent. In various embodiments, the additional antifungal agent is amphotericin B, caspofungin, anidulafungin or micafungin.

Screening Assays for Identifying a Compound that can Inhibit Pho84

In another aspect the invention provides a recombinant fungal cell comprising a polynucleotide encoding a reporter gene operably linked to a Pho84 promoter, wherein the fungal cell is hemizygous for Pho84. Without wishing to be limited by theory, inhibition of Pho84 prevents the activation of polynucleotides under the control of an operably linked Pho84 promoter. The reporter gene is repressed in environments where Pho84 is inhibited. Hemizygous Pho84−/+ strains are sensitized to Pho84 inhibitors. Therefore, recombinant fungal cells may be useful for identifying compounds that inhibit Pho84.

In another aspect, the invention provides a method of identifying a compound that inhibits Pho84 by exposing a recombinant fungal cell comprising a polynucleotide encoding a reporter gene operably linked to a PHO84 promoter to a putative inhibitor compound, wherein the fungal cell is hemizygous for Pho84; measuring the expression level of the reporter gene; and comparing the expression level of the reporter gene to a predetermined reference level; thereby identifying the compound as a Pho84 inhibitor. In various embodiments the reporter gene is green fluorescent protein, red fluorescent protein, β-galactosidase or chloramphenicol acetyltransferase. In various embodiments, when the level of the reporter gene is more than the predetermined reference level, it indicates that the compound is a Pho84 inhibitor. In another aspect the invention comprises a composition containing at least one Pho84+/− fungus cell and cell culture medium. This composition is useful for screening for compounds that may inhibit Pho84 due to the hemizygous strain's heightened sensitivity to Pho84 inhibition. In various embodiments, the composition further includes a putative Pho84 inhibitor. In various embodiments, the cell culture media is synthetic complete low phosphate media.

In a further aspect the invention comprises a method of identifying a compound that inhibits Pho84 by exposing at least one Pho84+/+ fungal cell and at least one Pho84+/− fungal cell to a putative inhibitor compound; and comparing a growth level of the at least one Pho84+/+fungal cell to a growth level of the at least one Pho84+/− fungal cell, thereby identifying the compound as a Pho84 inhibitor. In various embodiments, the at least one Pho84+/+ fungal cell and the at least one Pho84+/− fungal cell are grown on the same media. In various embodiments the growth level is determined by measuring colony size, OD₆₀₀, reduction of tetrazolium salts including but not limited to 2,3-bis-(2-methoxy-4-nitro-5-sulfophenyl)-2H-tetrazolium-5-carboxanilide (XTT) or the reduction of resazurin. Due to the heightened sensitivity of Pho84+/− cells to Pho84 inhibition the Pho84+/+ cells will exhibit higher growth levels than the Pho84+/− cells when exposed to a Pho84 inhibitor. This differential sensitivity may be most easily observed when the fungal cells are grown on the same media, especially low phosphate media.

### Compositions of the Invention

The invention provides a recombinant fungal cell comprising a polynucleotide encoding a reporter gene operably linked to a Pho84 promoter, wherein the fungal cell is hemizygous for Pho84. An isolated polynucleotide encoding a reporter gene operably linked to a Pho84 promoter is also provided. In particular, the isolated polynucleotide comprises the sequence of SEQ ID NO: 1:

CAAAAAATCTAATAATTACAAAACTTGTAAAGCATAAAACTAAAAACTTT
TTATTAATAAAAAGAAATATCTCGTTTGTATGTTAAATTGTTGTACACAT
GGGCAATTACTTGGATTGGCTAAACGAAATAATAAAAATGTTGCCATTCT
AGTTTTAAATACTTTATGATTTTAGATGCATTATTACATTTCGATTTAGT
CATGTAAATGATTAGTGTACCATGTTTGGTTGGTAATTGCCTCCCCATTC
AAGATAACTACACTCATGTCTTACCCAGACCTCCCTATATGCCACTCCAA
ACTATCCAACCCTTATAGAGATCACCCTTTTAATCCTCCAATTCATAGAT
TATTAACCTTGACATAGCTACCAGATATAAAATATTCAAATTTTTTTTGT
TCTCAACTTCACTCTATTCTCCATTTTTTACAGTTTACAGGAACATTCCC
AAAAGAAGCAAAAAAAAATAAAAATTGTAAGACTTGAATTACACAAAATA
AATTATAGTTTTTTTTTTTGCACTAAAATTCAATTTACACAAAATTTAAT
AATTTCTGAAAAGTATCAGTATCATCATCACCAAGAAAACGCAGCCTTCA
AATAAAAACTCATTTTAATTAAAACTCTCTTATGAAATTAAATTCATCCC
TAATAGAAAGGAAAGGAAAAAAAAAAAAAGAAATCTCTTGCATATCTCGT
TATATATTTTGTATGAAATAATATAATAAATATATACTTCAAATTAGAAT
ATATAAATATTTAAAACAATTCCCTTAAATTATTGAAATTTTTATTTTTT
TATAATTCTTCTTCTTCTTTTCTTTAAGGACTATTTCAATATCTCAATTG
TTATTTCACTTTATTTTCCTTCATATTTACTTATTCATTGGGTTTCCTGC
GTATATACATACCCTCCCCCCTCTTTTTGCTATATCACATTTACAACTAA
CACTATAGTTTTTACAAAAAGATGGTTGCTGAAATTCAARSCTCACACTA
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAG

where the C. albicans Pho84 promoter sequence is underlined and the reporter is GFP. In certain embodiments, the isolated polynucleotide has at least 90% or 95% sequence identity to SEQ ID NO: 1. In particular embodiments, the polynucleotide has at least 95% identity to SEQ ID NO: 1. In certain embodiments, the isolated polynucleotide encodes an isolated polypeptide comprising the amino acid sequence of SEQ ID NO: 2:

MSKGEELFTG VVPILVELDG DVNGHKFSVS GEGEGDATYG
KLTLKFICTT GKLPVPWPTL VTTFSYGVQC FSRYPDHMKQ
HDFFKSAMPE GYVQERTIFF KDDGNYKTRA EVKFEGDTLV
NRIELKGIDF KEDGNILGHK LEYNYNSHNV YIMADKQKNG
IKVNFKIRHN IEDGSVQLAD HYQQNTPIGD GPVLLPDNHY
LSTQSALSKD PNEKRDHMVL LEFVTAAGIT HGMDELYK.

The invention also provides an expression vector comprising the aforementioned polynucleotides positioned for expression in a cell. Cells comprising the expression are also provided. In certain embodiments, the cell is a fungal cell that is hemizygous for Pho84.

The invention also provides a composition comprising at least one Pho84+/− fungus cell and cell culture medium. One skilled in the art will appreciate that various types of suitable cell culture media can be used in accordance with the invention, including, but not limited to, those described in the foregoing figures and those described in the following examples. In certain embodiments, the composition further comprises a putative Pho84 inhibitor. In certain other embodiments, the compositions further comprises a Pho84+/+ fungus cell. In other embodiments, the cell culture medium comprise slow phosphate media.

Kits for Identifying a Compound that can Inhibit Pho84

In another aspect the invention provides a kit containing one or more recombinant fungal cells having a polynucleotide encoding a reporter gene operably linked to a Pho84 promoter, wherein the fungal cell is hemizygous for Pho84; a reagent for measuring the expression level of the reported gene; and written instructions describing the use of the kit including the steps of exposing the recombinant fungal cell to a putative inhibitor compound; measuring the expression level of the reporter gene; and comparing the expression level of the reporter gene to a predetermined reference level; thereby identifying the compound as a Pho84 inhibitor.

The kit instructs a user to practice the above described method for screening putative Pho84 inhibitors using a system including a reporter gene construct and contains the necessary components to facilitate doing so.

In another aspect the invention provides a kit comprising one or more Pho84+/+ fungal cells and one or more Pho84+/− fungal cells, a reagent for measuring the growth level of the fungal cells; and written instructions describing exposing at the Pho84+/+ fungal cell and the Pho84+/− fungal cell to a putative inhibitor compound; measuring a growth level for the at least one Pho84+/+ fungal cell and a growth level for the at least one Pho84+/− fungal cell; comparing a growth level of the at least one Pho84+/+ fungal cell to a growth level of the at least one Pho84+/− fungal cell, thereby identifying the compound as a Pho84 inhibitor.

The kit instructs a user to practice the above described method for screening putative Pho84 inhibitors by measuring different growth levels of Pho84+/+ and Pho84+/− fungal cells and contains the necessary components to facilitate doing so.

### Administration/Dosage/Formulations

The regimen of administration may affect what constitutes an effective amount. The therapeutic formulations may be administered to the subject either prior to or after the onset of fungal infection contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 1 μM and 10 μM in a mammal. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required.

In various embodiments, the compound may be administered topically, i.e. applied to the body surface as a liquid, ointment, gel etc., to treat superficial fungal infections like tinea capitis, tinea corporis, tinea cruris, tinea pedis.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, kidney function and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, topical or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., anti-fibrotic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In one embodiment, the tablets of the invention comprise saracatinib difumarate, mannitol, dibasic calcium phosphate anhydrous, crospovidone, hypromellose and magnesium stearate, with a film-coat containing hypromellose, macrogol 400, red iron oxide, black iron oxide and titanium dioxide.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation”. For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of a disease or disorder contemplated in the invention. Using a wax/pH-sensitive polymer mix, a gastric insoluble composition may be obtained in which the active ingredient is entrapped, ensuring its delayed release.

Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i. e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Materials and Methods.

The materials and methods employed in the following examples are here described.

Strains and Culture Conditions.

C. albicans strains were generated in the SN genetic background using HIS1 and ARG4 markers, as well as the CaNAT1 selectable marker, as described in. Two independent heterozygotes were used to derive homozygous null and reintegrant mutants of PHO84, as well as tetO-TOR1 mutants. Auxotrophies were complemented so that only prototrophic strains were compared in an experiment. Introduced mutations were confirmed by PCR spanning the upstream and downstream homologous recombination junctions of transforming constructs, and sequencing. Experiments with defined ambient Pi concentrations were performed in YNB 0 Pi (ForMedium Ltd, Norfolk, UK) with added KH₂PO₄ to stated concentrations. Other media known in the art was also used in various instances.

Screening Transposon Mutants for Altered Rapamycin Susceptibility.

The heterozygous mutant collection containing a mariner transposon marked with CaNAT1 (20) was used. Mutants were pre-grown at room temperature in 96-well plates containing 2xYPD with 8% glucose to minimize hyphal growth. Cells were replicated to YPD agar with vehicle (90% ethanol) or 20 ng/ml rapamycin. Clones showing less growth than the wild type (SC5314) were isolated as rapamycin hypersensitive. The transposon insertion site was identified by vectorette PCR (20).

Growth Assays.

For cell dilutions spotted onto agar media as previously described (20), saturated overnight cultures were diluted in 5-fold steps from an OD600 of 0.5. For growth curves in liquid media, saturated overnight cultures in YPD were washed once in 0.9% NaCl and diluted to an OD600 of 0.15 in 150 μl medium in flat bottom 96-well dishes. For growth assays including those during drug exposure, OD600 readings were obtained every 15 min in a plate reader and standard deviations of 3 technical replicates were calculated and graphed in Graphpad Prism. Growth during drug exposure was assayed in SC medium. Vehicle for Pho84 inhibitors PAA (Sigma, 284270) and Fos (Santa Cruz Biotechnology, # SC-253593A) was water and for amphotericin B (Sigma, A9528), DMSO. All panels shown represent at least 3 biological replicates.

Western Blots.

Cell harvesting, lysis, Western blotting and densitometry were performed as described in Chowdhury T & Köhler JR (2015) Ribosomal protein S6 phosphorylation is controlled by TOR and modulated by PKA in Candida albicans. Molecular Microbiology 98(2):384-402. At least three biological replicates were obtained for each experiment shown.

Hyphal Morphogenesis Assay.

Cells were revived from frozen stocks on solid YPD overnight, washed and resuspended in 0.9% NaCl to OD600 0.1. Variations between single colonies and colony density effects were minimized by spotting 3 μl cell suspension at 4 or 6 equidistant points, using a template, around the perimeter of an agar medium plate as in (20). For small molecule Pho84-inhibitor effects on hyphal formation, Spider and RPMI were used (TOKU-E, Cat # R8999-04), the latter with 0.22 mM KH2PO4 buffered to pH 7 with 50 mM MOPS. All panels shown represent at least 3 biological replicates.

Acid Phosphatase Assays.

Overnight cultures in SC were diluted to an OD600 0.05 into YNB medium buffered to pH 4 with 50 mM sodium citrate containing 0 or 11 mM KH2PO4 and grown overnight. P-Nitrophenyl Phosphate (Sigma, N4645) was added to washed cells to a concentration of 5.62 mg/ml. After 15 mins at room temperature the reaction was stopped with Na₂CO₃ (pH=11) to a concentration of 0.3 g/ml, and OD₄₂₀ and OD₆₀₀ were measured. At least 3 biological replicates, with 3 technical replicates each, were obtained.

Intracellular Pi Assays.

Free and total Pi was measured by colorimetric molybdate assay. Cultures were washed with distilled water twice, resuspended in 500 μL 0.1% Triton X-100, and lysed by glass bead homogenization. Lysate protein content was determined using a BioRad Protein Assay kit. Free Pi was measured in unboiled lysate, then total phosphate was measured after boiling 3-30 μg of whole cell lysate for 10 min in 1 N H2SO4. At least 3 biological replicates, with 3 technical replicates each, were obtained.

RT-PCR Expression Analysis.

Cells were grown overnight in YPD medium with 5 ng/ml doxycycline, diluted into YPD with 30 μg/ml doxycycline, and harvested at time 0, 2 h and 4 h. RNA was extracted with the Direct-Zol RNA miniprep kit (Zymo Research # R2051).

### Example 1: A Screen of Haploinsufficient Transposon Mutants for Altered Rapamycin Susceptibility Identified a PHO84 Ortholog

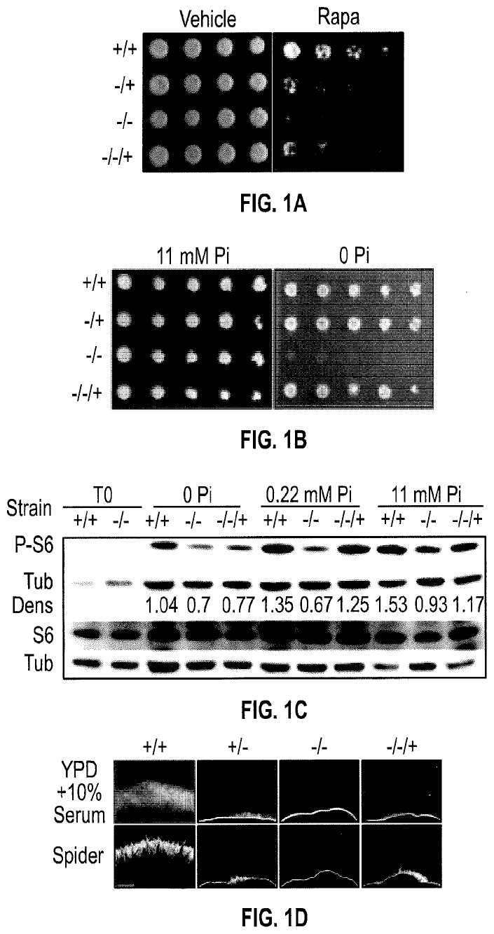

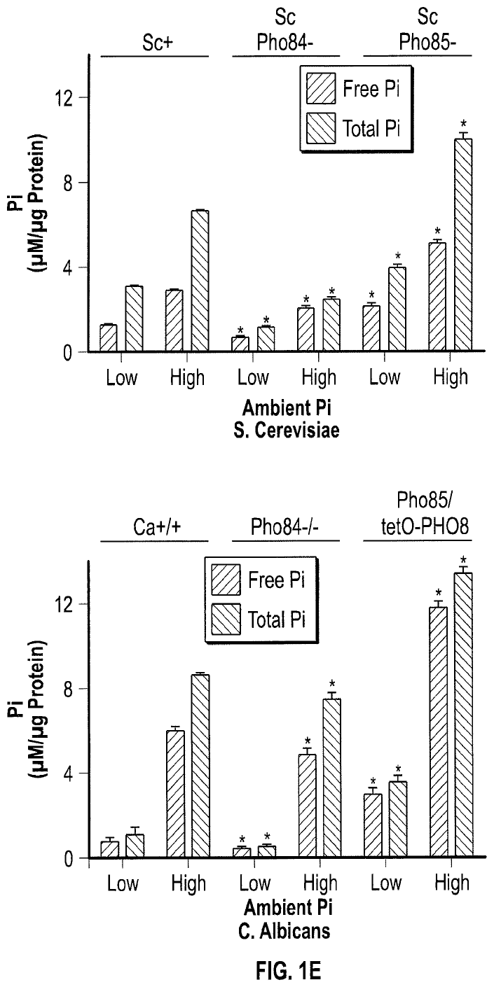

A heterozygous mutant collection of mariner-transposon insertions marked with the dominant selectable marker NAT1 was screened for altered rapamycin susceptibility. A transposon mutant hypersensitive to rapamycin was isolated in which the transposon disrupts the promoter of orf19.655, 67 bp upstream of the predicted translational start site (FIG. 6A). This orf encodes a protein with 66% amino acid identity to S. cerevisiae Pho84 and 55% amino acid homology to the Piriformospora indica PiPT phosphate transporter whose crystal structure was recently described (FIG. 6B). According to Candida Genome Database (CGD) nomenclature, this orf was called C. albicans PHO84, and used the CGD sequence for further analysis. To confirm that rapamycin hypersensitivity of the transposon mutant was linked to the disrupted PHO84 locus, two independent heterozygous deletion mutants and their homozygous null derivatives were constructed. Mutant phenotypes in these two lineages were the same, and one was chosen for further characterization (FIG. 6C). These mutants were also rapamycin hypersensitive (FIG. 1A), confirming that PHO84 is required for normal tolerance of rapamycin. The cytoplasmic membrane protein Pho84 is the major highaffinity phosphate (Pi) transporter in S. cerevisiae. PHO84 expression is controlled by the PHO regulon, a homeostatic system that maintains Pi availability for metabolism and growth in fluctuating external Pi conditions. C. albicans pho84 mutants, like those in the S. cerevisiae homolog, failed to grow on medium without inorganic phosphate (FIG. 1B). Heterozygous and re-integrant cells, apparently haploinsufficient for rapamycin tolerance (FIG. 1A), grew robustly on 0 Pi medium (FIG. 1B), indicating that mechanisms other than haploinsufficiency affect growth during Pi depletion, like the feedback loops between expression of high- and low-affinity Pi transporters characterized in detail in the S. cerevisiae PHO regulon. In liquid media with 1 mM Pi, growth of pho84 cells was close to wild type (FIG. 6D). Expression of the C. albicans PHO84 homolog restored growth on medium lacking Pi to S. cerevisiae pho84 mutants (FIG. 6E), indicating functional orthology. Wild type cells secrete acid phosphatase in response to low ambient Pi to mobilize covalently bound Pi from their environment, and this response was used for decades in studies of the S. cerevisiae PHO regulon. C. albicans pho84−/− mutants, like those in S. cerevisiae, inappropriately de-repressed acid phosphatase secretion in high ambient Pi (FIG. 6F), consistent with a conserved role of C. albicans Pho84 in the PHO regulon.

### Example 2: C. albicans Pho84 is Required for the Normal TORC1 Response to Pi Availability

The relationship between Pho84 and TORC1 was then examined. To test whether rapamycin hypersensitivity is due to decreased TORC1 kinase activity in the pho84−/− mutant, the phosphorylation state of ribosomal protein S6 (P-S6) was monitored, which Applicant previously showed is controlled by TORC1 signaling. Null mutants in PHO84 had a weaker P-S6 signal than wild type during Pi refeeding at every Pi concentration of the media, though they responded to increasing Pi concentrations with an increasing P-S6 signal (FIG. 1C). Pho84 therefore is required for normal anabolic TORC1 signaling, and TORC1 activity responds to ambient Pi availability.

### Example 3: Heterozygous and Homozygous Deletion Mutants in PHO84 are Defective in Hyphal Morphogenesis

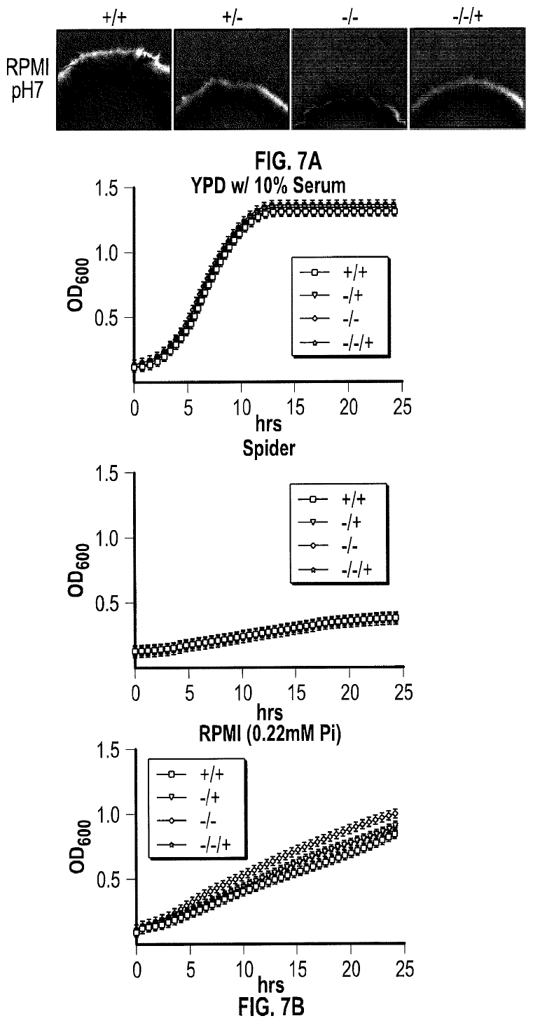

TORC1 regulates hyphal morphogenesis in C. albicans, an important virulence determinant. Hyphal morphogenesis was defective in pho84 mutants on YPD agar medium with 10% serum, Spider medium and RPMI 1640 (FIGS. 1D and 7A), while mutant and wild type grew equally in these media when the hyphal temperature signal was absent (FIG. 7B). While many signaling pathways converge on morphogenesis, these findings are formally consistent with defective regulation by TORC1 (2).

### Example 4: Pi Content of Cells Lacking Pho84 is Diminished

It was questioned if C. albicans TORC1 activity may be downregulated in response to decreased intracellular Pi in pho84 mutants, analogously to the response of S. cerevisiae TORC1 to decreased intracellular amino acids. Using pho85 mutants as controls known to hyperaccumulate intracellular Pi, it was found that intracellular Pi concentrations were lower in pho84−/− null than in wild type cells in low and high Pi-containing media, though the difference was substantially less than in the homologous S. cerevisiae mutant-wild type pair (FIG. 1E). Diminished intracellular Pi concentrations of C. albicans pho84−/− cells may be responsible for the decreased TORC1 activation state, possibly in addition to lack of a putative TORC1-activating function performed specifically by Pho84.

### Example 5: Gtr1 Links Pho84 to TORC1 in C. albicans

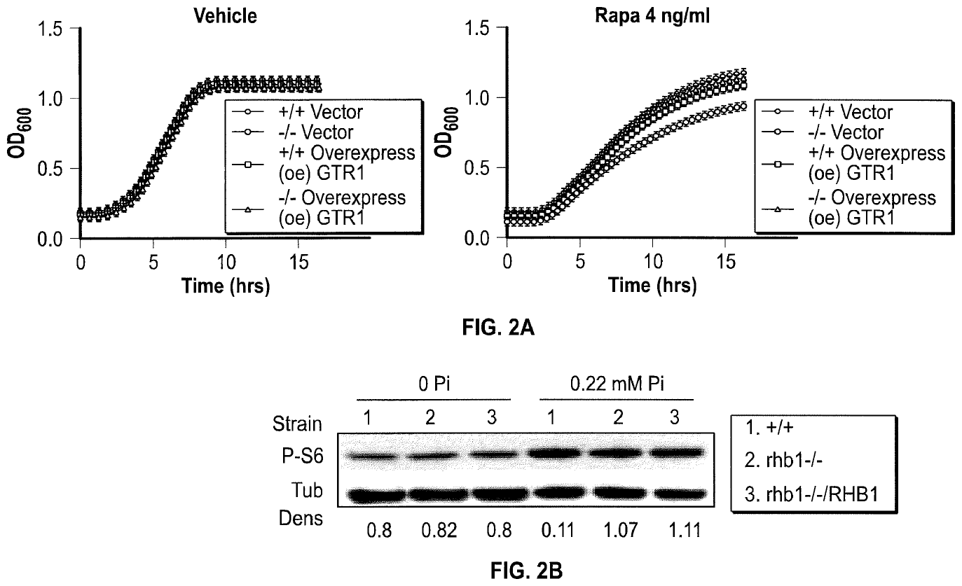

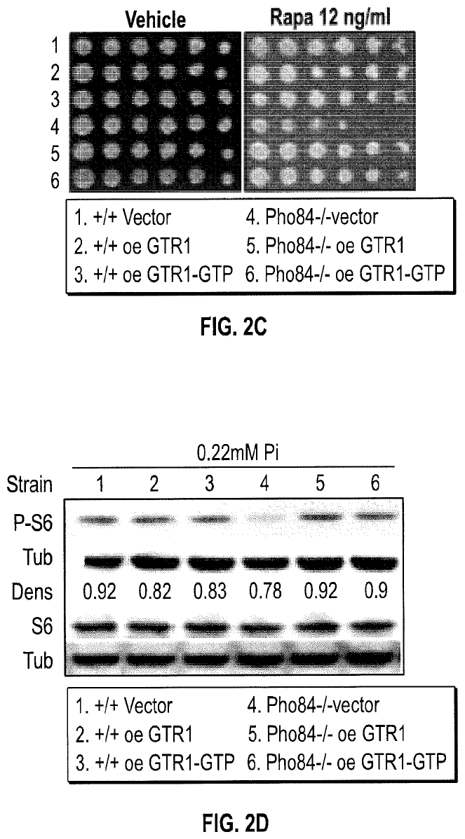

Seeking a molecular link between Pho84 and TORC1 activity, the possibility that Gtr1 may connect Pho84 to TORC1 was considered. GTR1 was first described for its functional and physical proximity to S. cerevisiae PHO84, and its product later was characterized as a component of the TORC1-activating EGO complex. It was found that the P-S6 response of gtr1−/− cells to phosphate refeeding was blunted. To determine whether this is an unspecific effect of decreased upstream TORC1 signaling, mutants in another small TORC1-activating GTPase, RHB1, were tested. rhb1 mutants responded to Pi refeeding like the wild type (FIG. 2A), suggesting that a Pi signal to TORC1 is transmitted specifically through Gtr1.

If Gtr1 acts downstream of Pho84 in activating TORC1, its overexpression may suppress pho84−/− phenotypes. GTR1 was overexpressed from the ACT1 promoter in wild type and pho84−/− C. albicans cells. Compared with rapamycin hypersensitive pho84−/− cells transformed with the empty vector, the resulting pho84−/− pACT1-GTR1 cells showed wild type tolerance to rapamycin, suggesting recovery of their TORC1 signaling activity (FIG. 2B, C).

To investigate this possibility TORC1 activity was tested directly by comparing the P-S6 signal of pho84−/− cells transformed with the empty vector with that of pho84−/− pACT1-GTR1 cells. Overexpression of GTR1 recovered Rps6 phosphorylation in pho84−/− cells nearly to wild type levels (FIG. 2D). A GTR1 mutant encoding constitutively GTP-bound Gtr1Q67L, homologous to S. cerevisiae Gtr1Q65L, was then constructed and overexpressed from the ACT1 promoter. This GTR1-GTP allele suppressed the TORC1 signaling defect of pho84−/− cells apparently equally to the overexpressed wild type GTR1 (FIGS. 2C, D). Overexpression of GTR1 or GTR1-GTP did not increase phosphorylation of S6 in wild type cells (FIG. 2D). These findings are consistent with the model that in C. albicans, Gtr1 indirectly or directly conveys a Pi signal to TORC1 and links Pho84 to TORC1 signaling.

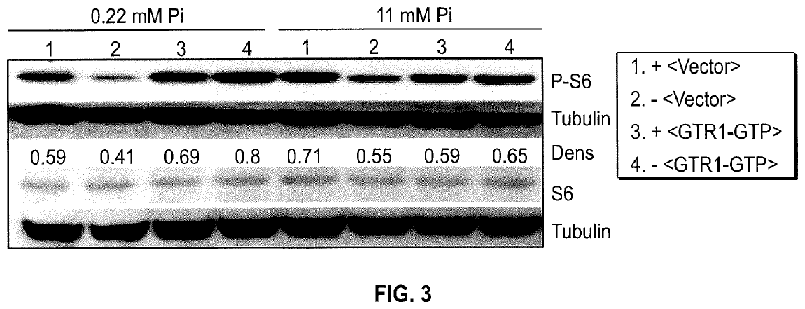

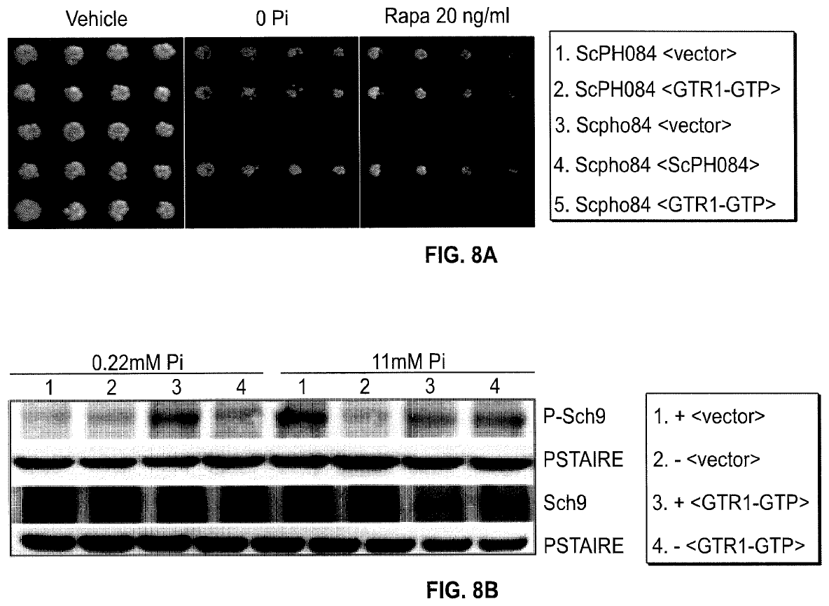

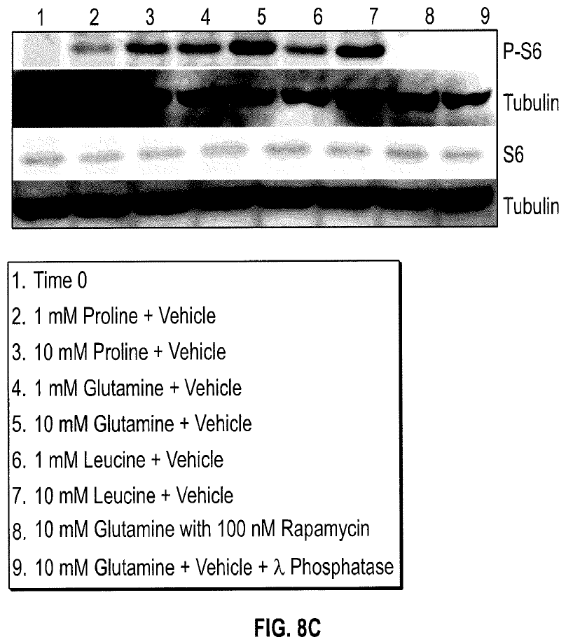

The relationship of Pho84 to TORC1 activity in the model yeast S. cerevisiae was examined. A pho84 null mutant in the S288C genetic background was hypersensitive to rapamycin (FIG. 8A) at an intermediate ambient Pi concentration (1 mM). Of note the rapamycin phenotype was highly responsive to the Pi concentration of pregrowth media. Rapamycin tolerance was not decreased by loss of PHO84 in the Σ1278b background. Rapamycin hypersensitivity was not suppressed, but the S. cerevisiae Sch9 phosphorylation state (FIG. 8B) and P-S6 signal intensity, which in S. cerevisiae also responds to TORC1 activation (FIGS. 3 and 8C), were recovered by constitutively active Gtr1 in pho84 null cells. These findings suggest that Pi homeostasis and TOR signaling are linked in S. cerevisiae as in C. albicans, though specific molecular connections seem to have divergently evolved in these two fungi.

### Example 6: TORC1 Modulates the PHO Regulon

As TORC1 not only responds to nutrient availability, but also directs nutrient uptake e.g. by regulating expression of amino acid and ammonium transporters, it was questioned whether it may play a similar role in phosphate acquisition. Given known discrepancies between rapamycin exposure and physiological TOR modulation, this potential connection was examined genetically. Repressible tetO was used to control expression of C. albicans TOR1 or of a hypomorphic TOR1Δ1-381 encoding a protein lacking the first 381 amino acids which form protein-protein interaction HEAT repeat domains. The effect of TOR1 depletion on expression of PHO84 was then examined.

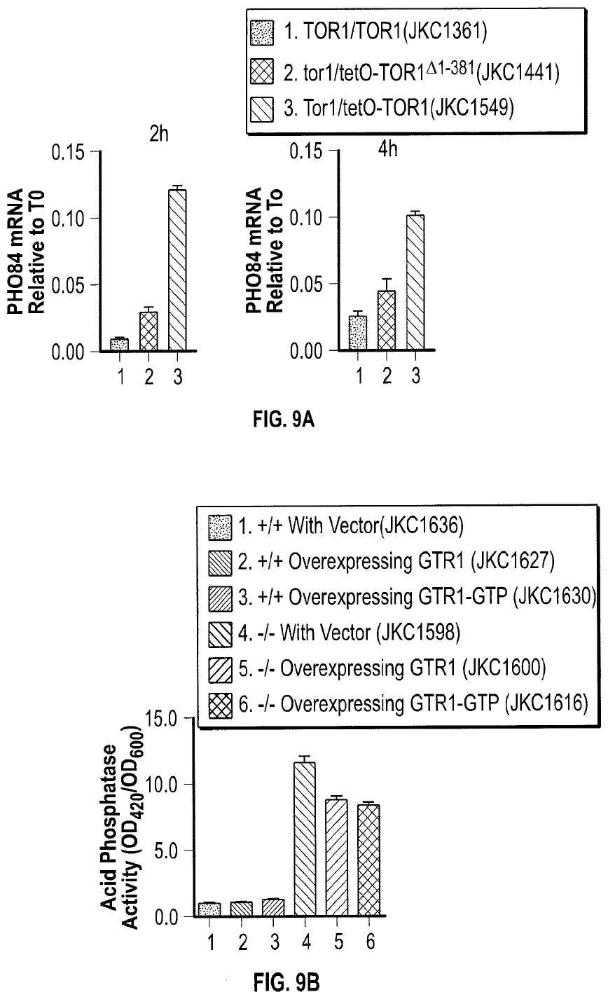

When wild type cells were transferred from overnight cultures into fresh rich medium, PHO84 mRNA levels dropped, in accordance with the PHO regulon's response to availability of fresh Pi sources. In cells depleted of either the wild type or the N-terminally truncated TOR1 allele, PHO84 expression also decreased but to a significantly lesser extent (FIG. 9A). Full length TOR1 permitted greater PHO84 expression than the TOR1Δ1-381 allele, suggesting structural perturbation of TORC1 by truncation of Tor1 affects its inhibitory as well as activating functions. Active TORC1, signaling nutritional repletion, hence contributes input to the PHO regulon to downregulate Pi starvation responses, while loss of TORC1 activity conveys a starvation signal to dampen these responses (FIG. 9A). Similarly, overexpression of Gtr1 and Gtr1-GTP blunted upregulation of secreted acid phosphatase in pho84−/− cells (FIG. 9B), supporting the model that in response to Pi TORC1 signaling downregulates the PHO regulon to integrate its activity with availability of other nutrients.

### Example 7: Small-Molecule Inhibitors of Pho84 Repress TORC1 and Potentiate Antifungal Activity

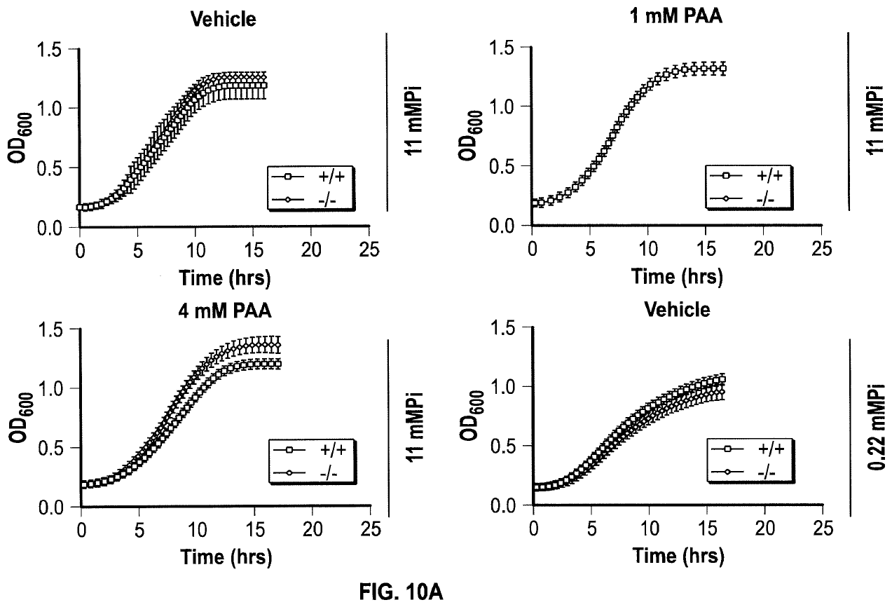

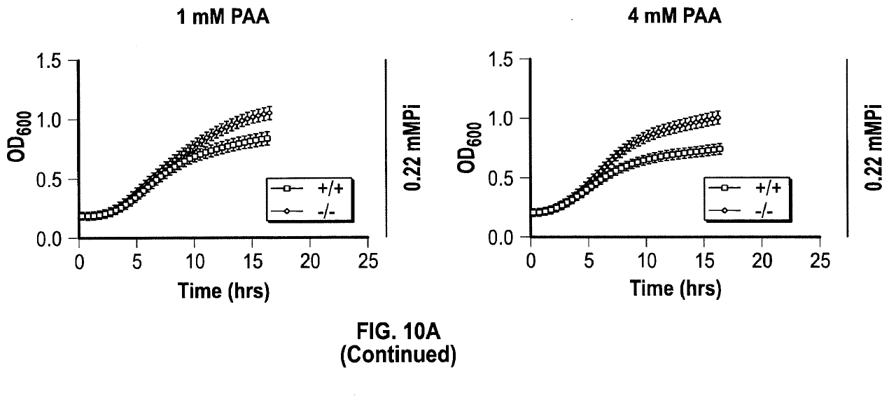

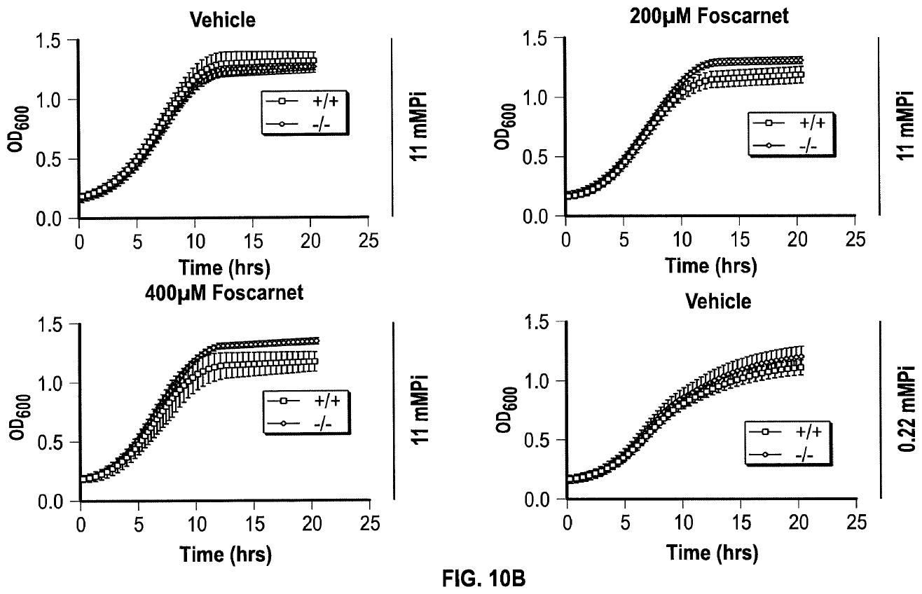

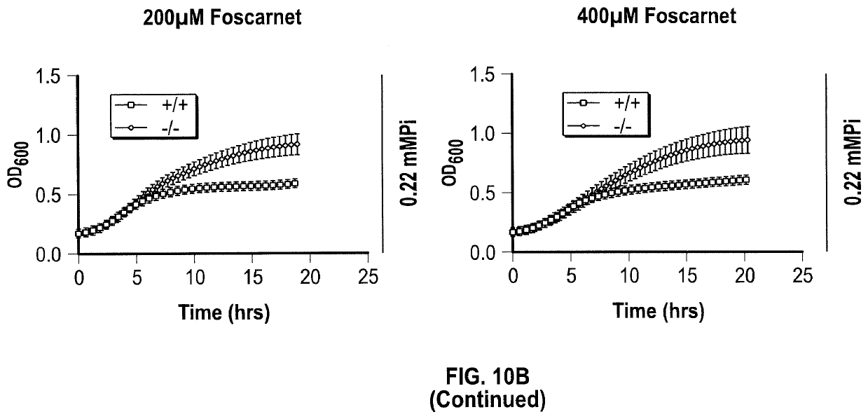

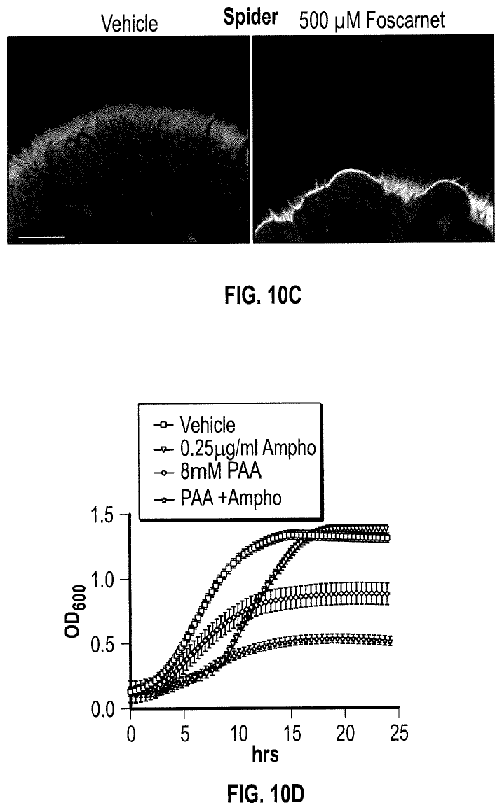

S. cerevisiae Pho84 has been characterized as a Pi transceptor signaling to PKA, through identification of point mutations and small molecules that preferentially perturb transport, signaling or both. Direct pharmacological inhibition of C. albicans TORC1 with rapamycin incurs too high a cost on host immune function to be clinically useful. In order to determine whether blocking Pho84 with its small-molecule inhibitors phosphonoformic acid (foscarnet, Fos) and phosphonoacetic acid (PAA), which inhibit C. albicans growth in dependence on the presence of their target Pho84 (FIG. 10, panels A, B), can indirectly inhibit C. albicans TORC1. Exposure of wild type cells to the FDA approved antiviral foscarnet inhibited Rps6 phosphorylation (PS6) in a dose-dependent manner (FIG. 4A), at Fos concentrations attained in human plasma during antiviral therapy. In heterozygous cells (pho84−/+), whose haploinsufficiency phenotypes likely reflect decreased copies of the drug target, Pho84, P-S6 was hypersensitive to Fos (FIG. 4A). In cells lacking the target Pho84, exposure to Fos did not further decrease the P-S6 signal (FIG. 4A). Pho84 inhibition with small molecules also recapitulated the hyphal growth defect seen in cells genetically depleted of PHO84 (FIGS. 1D, 4B and 10C).

Potentiating existing antifungals is a promising strategy. Fos at concentrations reached in plasma during antiviral therapy, and PAA, potentiate activity of the antifungal amphotericin B, at concentrations of the latter far below those in serum or tissue during standard dosing (FIG. 4C, 10D). Activity of the antifungal micafungin, belonging to the distinct drug class of echinocandins, was also potentiated (FIG. 4D). Because Pho84 is not conserved in mammals, inhibition of Pho84 offers a novel approach to fungal-specific TORC1 inhibition (FIG. 5) and to antifungal potentiation, as shown in the proof-of-principle experiments with PAA and Fos.

### Example 8: Pho84 is Required for Virulence of C. albicans in a Wild-Type Drosophila Model

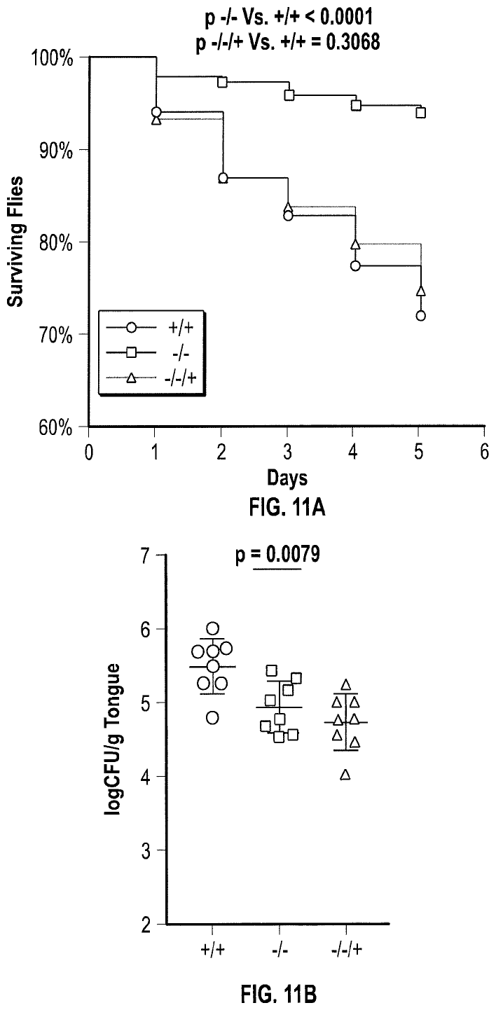

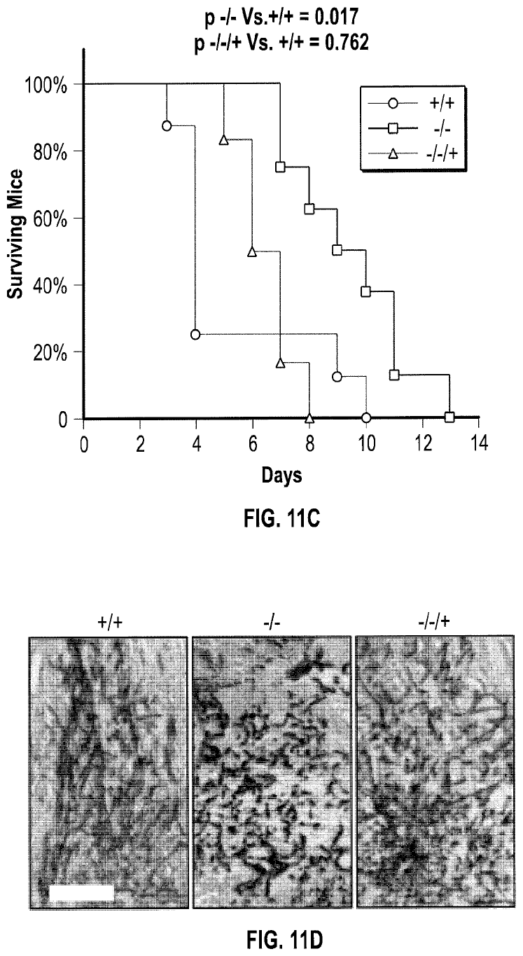

Null mutants in PHO84 are defective in TORC1 signaling and in hyphal growth. As C. albicans hyphal growth is a virulence determinant, pho84−/− null mutant cells' virulence in a Drosophila model was examined. In the wild type OregonR strain used, Drosophila immune responses are intact, so that the fungus must contend with the full complement of innate immunity. By 5 days after infection, 30% of flies injected with PHO84 wild type, and 8% of those injected with pho84−/− null cells had died (p-value <0.001). Heterozygous −/+ pho84/PHO84 and reintegrant −/−/+ pho84/pho84/PHO84 mutants had intermediate phenotypes that were statistically indistinguishable from null mutants and wild type, respectively (FIG. 11A). Hence, in a whole animal model of simple innate immunity, Pho84 is required for virulence. The data is presented in FIG. 11.

### Example 9: Pho84 Contributes to Virulence in Murine Disseminated and Oropharyngeal Candidiasis

Given their virulence defect in an insect model, the virulence of pho84−/− null cells in two murine models of infection was examined. One path of natural C. albicans infection is invasion of the mucosa, which is not modeled in the fruit fly. Pho84−/− null and wild type cells were compared for the ability to proliferate in a murine oropharyngeal candidiasis model. Loss of Pho84 resulted in decreased fungal burden in tongue tissue (FIG. 11B), indicating that in this model, functions of Pho84 contribute to virulence during oropharyngeal infection.

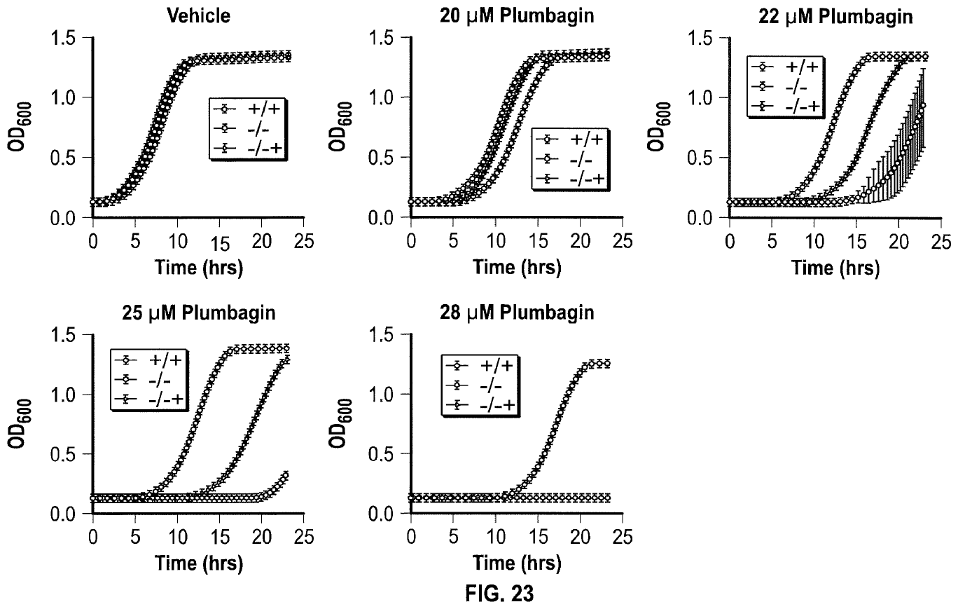

In the oropharyngeal candidiasis model, loss of virulence of the pho84−/− null cells and pho84−/−/+ reintegrant cells were similar. Haploinsufficiency of PHO84 heterozygous and reintegrant cells was observed. In fact, in a single phenotype, hypersensitivity to oxidative stress, Applicants observed haploinsufficiency or tolerance recovery to nearly wild type levels in reintegrants, depending on the intensity of the stress (FIG. 23).

Once C. albicans cells have crossed the mucosa and entered the bloodstream, they disseminate to distant organs and initiate metastatic foci of infection. Therefore, it should be determined whether Pho84 is required for C. albicans virulence during hematogenous infection. Survival of mice injected intravenously with wild type, pho84−/− null and PHO84 reintegrant cells was compared. As shown in FIG. 11C, more than 70% of the mice infected with the wild type strain died within 4 days of infection. Loss of Pho84 extended survival to 12 days in this model. The morphogenetic state of infecting filamentous cells were assessed according to these criteria: filaments with parallel-sided walls with no constrictions and few branches were categorized as hyphae, while those with constrictions at the septa and regular branching were defined as pseudohyphae. Gomori-Methenamine Silver stained sections of kidneys from moribund mice showed wild type cells growing predominantly in the hyphal form with interspersed yeast cells (FIG. 11D). In contrast, pho84−/− null cells were a mixture of pseudohyphal filaments and yeast with occasional hyphae, as judged from the appearance of the filaments (FIG. 11D). Hence, a hyphal morphogenesis defect previously observed in vitro may contribute to loss of virulence of pho84−/− null cells in vivo.

### Example 10: Pho84 is Required for Tolerance of Whole Blood Candidacidal Activity, Neutrophil Killing and Reactive Oxygen Species (ROS) Exposure

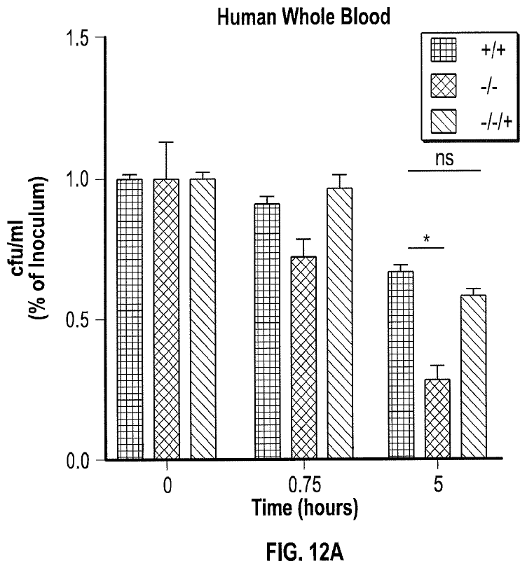

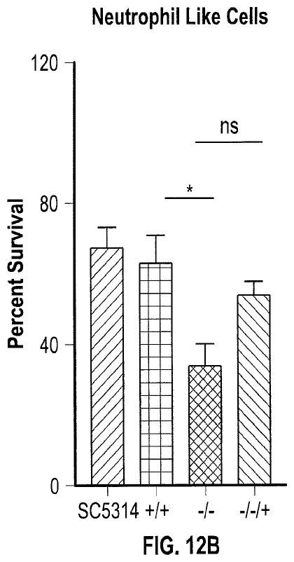

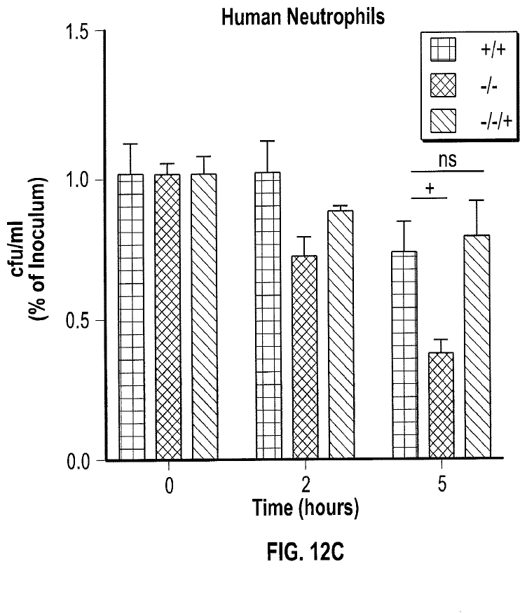

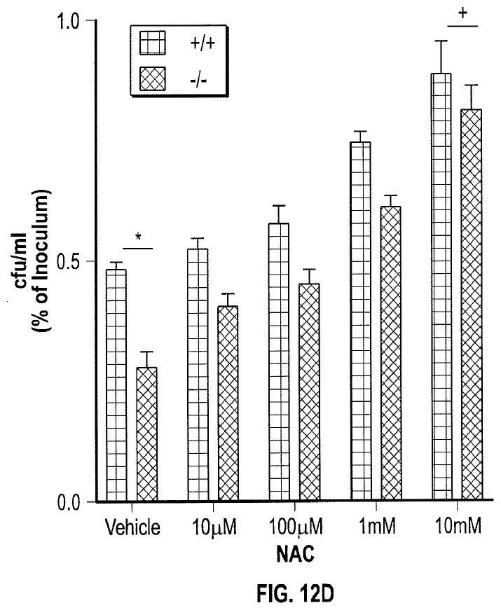

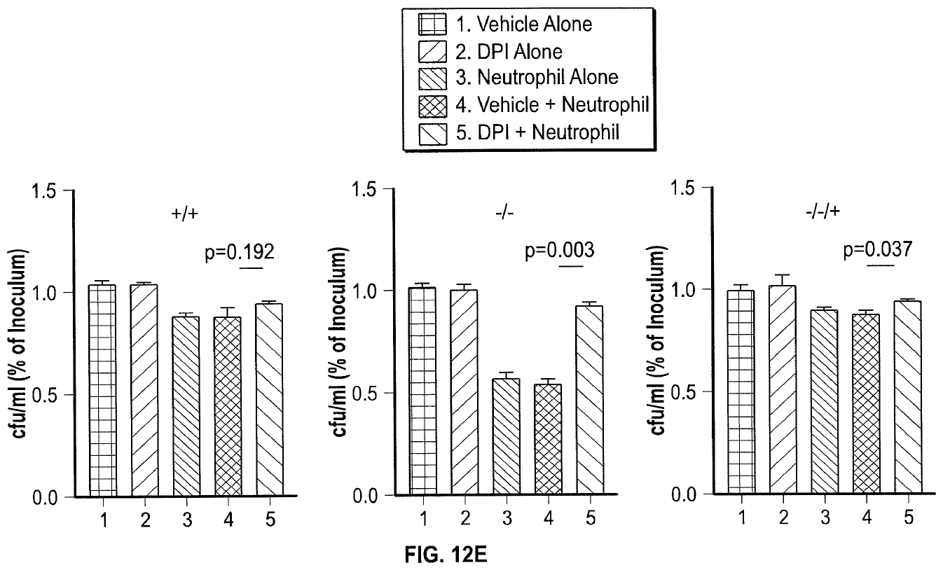

During bloodstream infection, invading C. albicans cells encounter host blood components. Heparinized whole blood from healthy human volunteers was incubated with C. albicans and survival of pho84−/− null cells was found to be significantly decreased after 5 hours, compared with the wild type and reintegrant (FIG. 12A). This finding indicates that Pho84 has a role in C. albicans' tolerance of whole blood candidacidal activity.

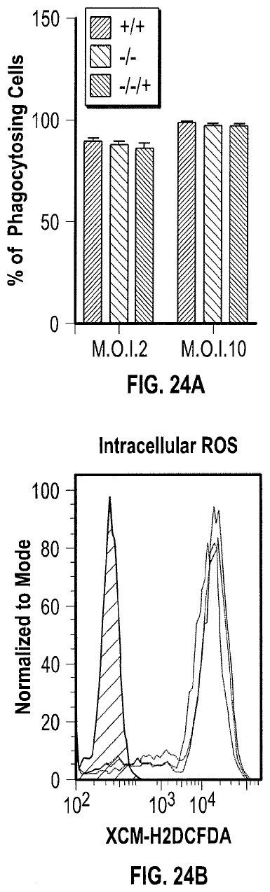

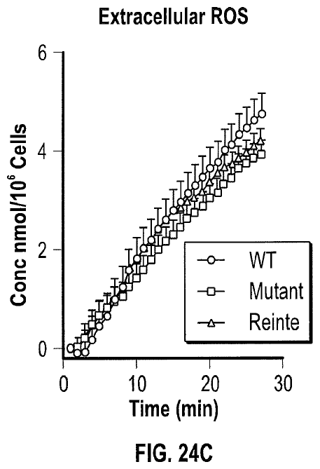

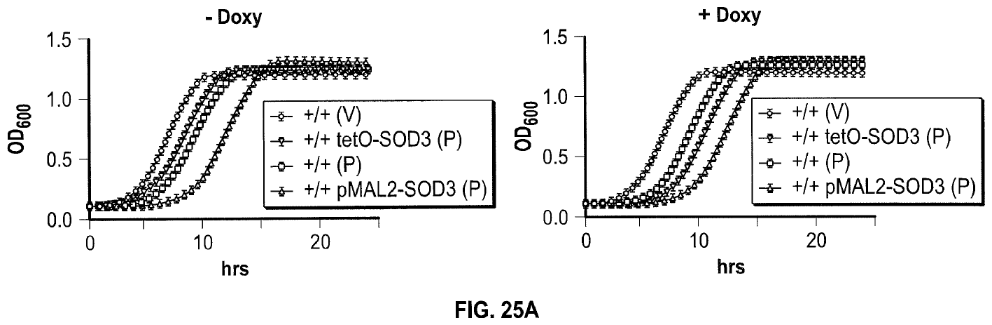

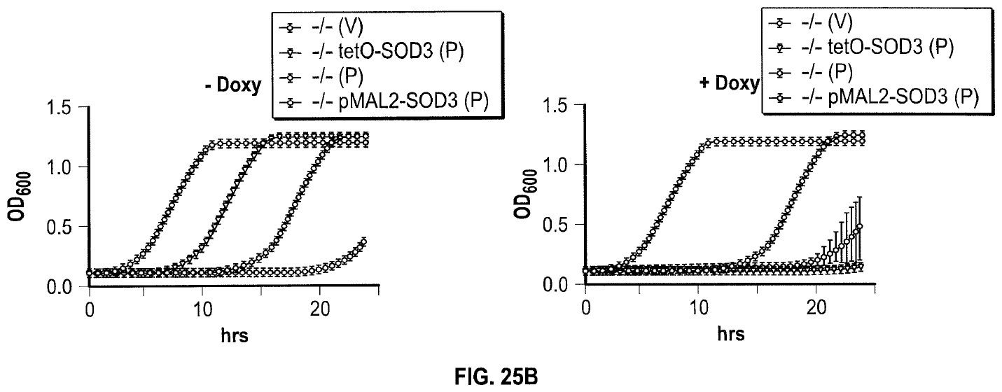

Neutrophils are the critical elements of cellular innate immunity with a major role in the first line of defense against invasive candidiasis. Therefore, the ability of pho84−/− null cells to survive the attack of human neutrophils was tested. The HL-60 human promyelocytic leukemia cell line which can be induced to differentiate into neutrophil-like cells was used. Null mutants in PHO84 were significantly more sensitive to killing by these neutrophil-like cells than wild type or reintegrant cells (FIG. 12B). This finding was confirmed in primary neutrophils isolated from healthy human donors (FIG. 12C). To distinguish hypersensitivity of pho84−/− null cells to neutrophil cidal activity from their putatively increased phagocytic uptake, phagocytosis assays were performed and found that pho84−/− null and wild type cells were equally taken up by the neutrophils (FIG. 24A). Similarly, it was examined whether pho84−/− null mutants may exert an increased stimulus toward ROS generation on neutrophils, since ROS production is a major neutrophil candidacidal mechanism. No difference was found in intracellular or extracellular ROS production between neutrophils that had phagocytosed wild type or reintegrant cells, and those that had phagocytosed pho84−/− null mutant cells (FIG. 24, panels B and C). These findings suggest that Pho84 contributes to protecting C. albicans against neutrophil killing.

To examine whether in fact ROS are responsible for pho84−/− null cells' increased susceptibility to neutrophils' cidal activity, Candida-ingesting neutrophils were treated with the ROS-scavenging compound N-acetyl cysteine (NAC). In a dose-dependent manner, NAC rescued hypersensitivity of pho84−/− null cells to neutrophil killing (FIG. 12, panel D). To block ROS production a priori, neutrophil NADPH oxidase (NOX), the enzyme complex responsible for generating the ROS oxidative burst, was inhibited by preincubating neutrophils with diphenyliodonium (DPI) and survival of C. albicans cells was followed. Inhibition of neutrophil NOX abolished hypersensitivity of pho84−/− null cells to neutrophil killing (FIG. 12E), supporting the idea that Pho84 is required for tolerance specifically of ROS-mediated neutrophil candidacidal activity.

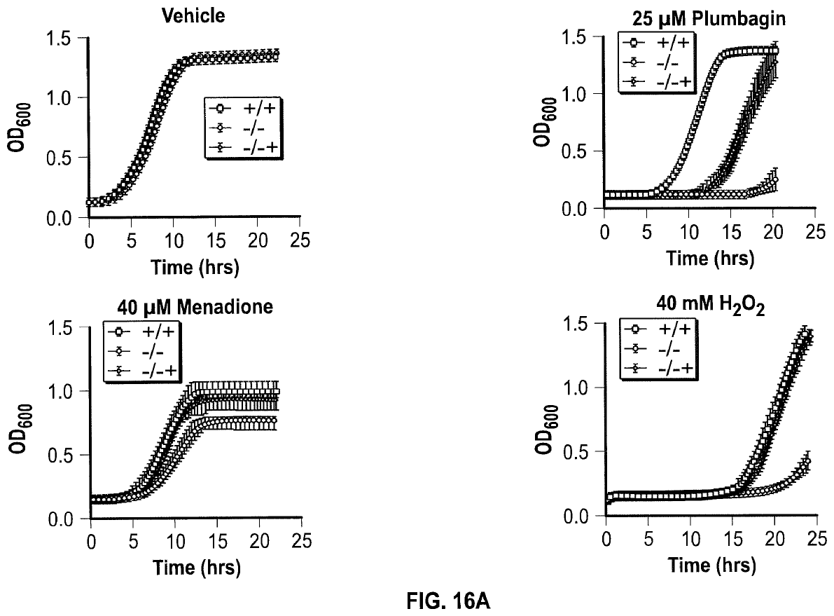

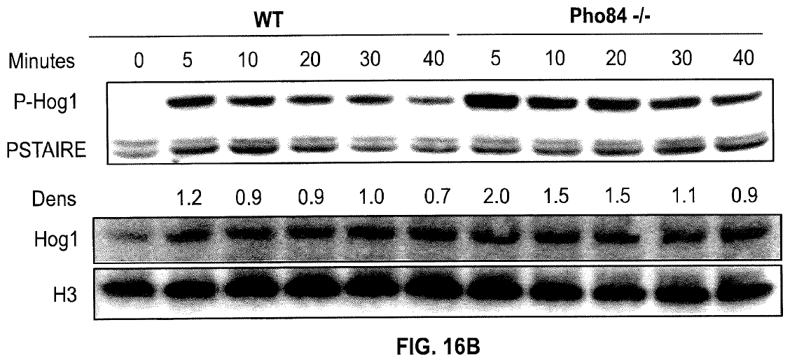

These findings raised the question whether pho84−/− null mutants are simply hypersensitive to ROS. Exposing wild type, pho84−/− null, and reintegrant cells to the inducers of superoxide anion, plumbagin and menadione, as well as hydrogen peroxide (H₂O₂), it was found that each of these compounds inhibited growth of the mutant more strongly than that of the wild type (FIG. 16A). Hypersensitivity of pho84−/− null cells to neutrophil killing may therefore be due to their hypersensitivity to oxidative stress.

### Example 11: Pho84 is Required for ROS Management

Defective HOG pathway signaling might be responsible for the inability of pho84−/− null cells to manage oxidative stress. The HOG pathway is a major signaling system by which C. albicans induces survival responses to RO. The phosphorylation state of the central kinase of the pathway, Hog1, was examined as a readout of HOG activation in response to oxidative stress. It was found that contrary to expectations, pho84−/− null cells showed prolonged and hyperintense Hog1 phosphorylation during a timecourse of peroxide-mediated induction (FIG. 16B).

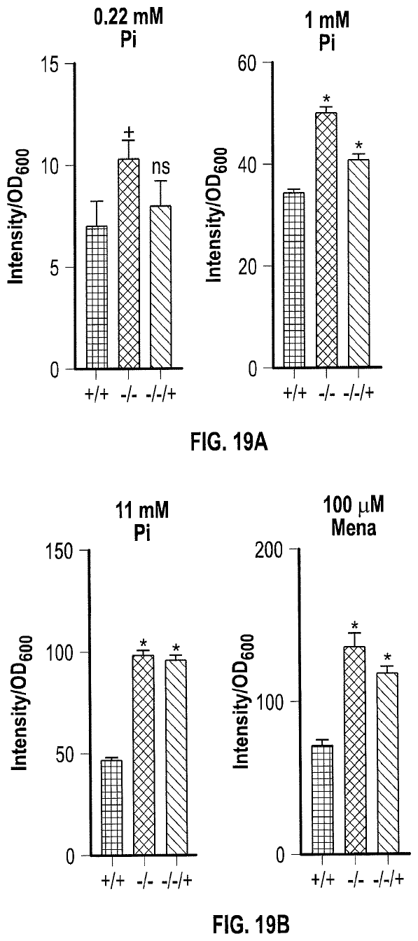

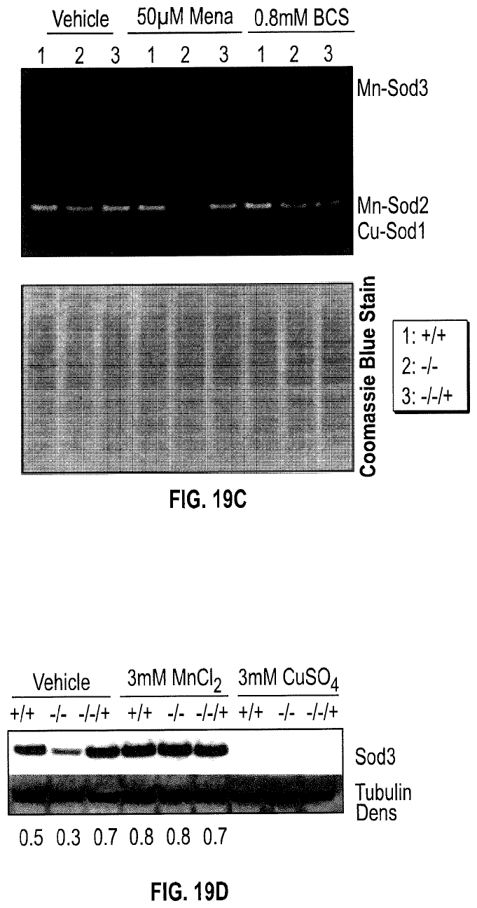

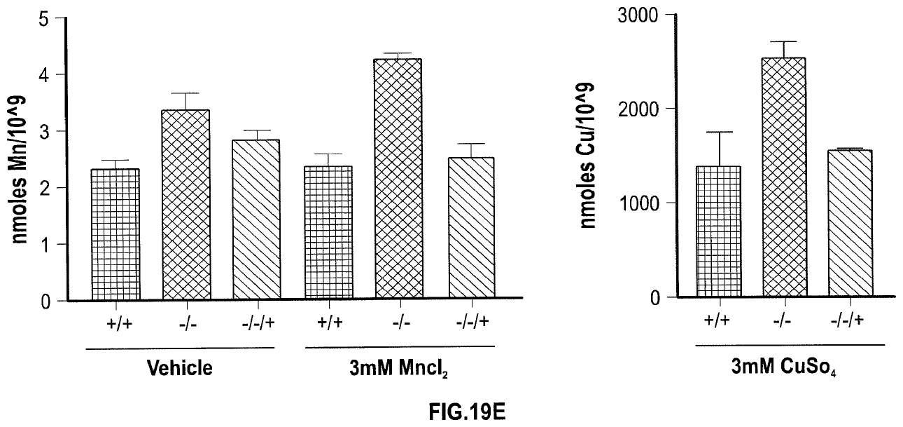

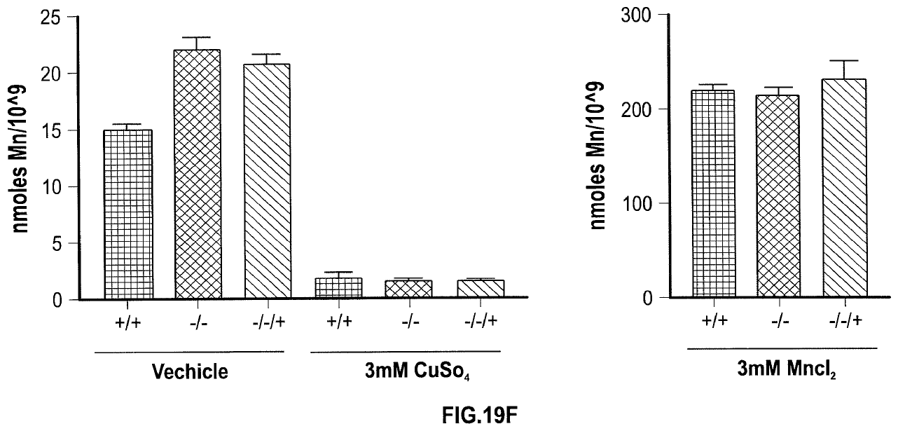

The apparent paradox of Hog1 pathway hyperactivation of cells lacking Pho84, and their increased susceptibility to extrinsic ROS, might be reconciled if these cells are unable to manage intracellular ROS. ROS are intrinsically generated by normal mitochondrial respiration. The DCFDA detectable ROS of unstressed exponentially growing pho84−/− null and wild type cells were compared, and of these cells exposed to oxidative stressors. Cells devoid of Pho84 had an increased ROS content, compared to the wild type, when unexposed or exposed to the superoxide anion-generating compound menadione, or to peroxide-generating H₂O₂ (FIGS. 19A and 19B). Their inability to manage ROS was not due to starvation for inorganic phosphate (Pi), because in Pi-replete media, which induced higher ROS contents of all strains presumably due to increased metabolic activity, pho84−/− null cells still contained significantly more ROS (FIG. 19A). It was concluded that Pho84 has a role in ROS management in C. albicans.

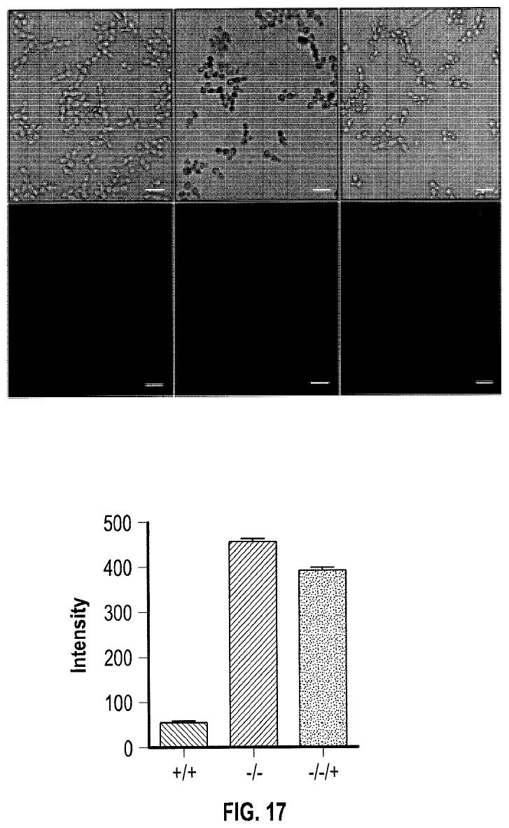

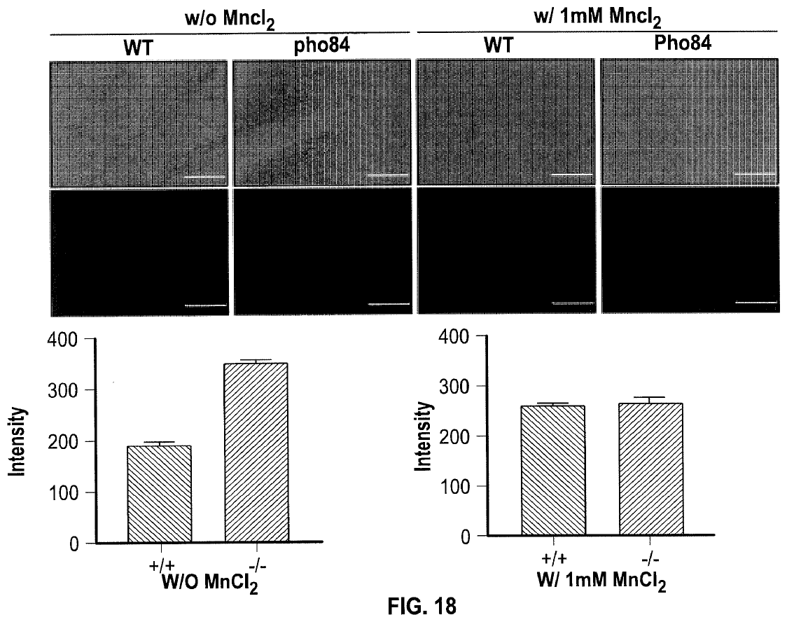

Furthermore, loss of Pho84 leads to accumulation of ROS (FIG. 17). However, accumulation of ROS in Pho84−/− null cells can be partially rescued by increased ambient Mn concentration (FIG. 18)

### Example 12: Superoxide Dismutase Expression is Perturbed in Pho84−/− Null Cells

Superoxide dismutates (SODs) contribute to ROS management by disproportionating the superoxide anion into H₂O₂ and oxygen, using a redox-active metal. Among the 3 intracellular and 3 extracellular SODs of C. albicans, manganese-using Sod3 enables C. albicans to switch between SODs depending on ambient copper concentrations during infection. In C. albicans pho4 null mutants, lacking the DNA binding protein that controls the PHO regulon, mRNA expression of 3 copper-using superoxide dismutases (SOD1, SODS and SOD6) is upregulated, while that of manganese-using SOD3 is decreased. It was questioned whether increased ROS content in pho84−/− null cells might be due to decreased SOD protein content or activity. pho84−/− null cells showed a subtle decrease of Sod1 activity (FIG. 19C), as assayed by nitroblue tetrazolium (NBT) reduction in non-denaturing protein gels, which display activity of the abundant 2 intracellular SODs, Sod1 and 2. Since under most conditions, Sod3 activity is not detectable on these gels, its protein abundance was examined by Western blot. Sod3 protein concentration was markedly reduced in pho84−/− null cells (FIG. 19C) in the absence of oxidative stress. In the presence of high ambient manganese, Sod3 concentrations in the pho84 mutant appeared as robust as those in the wild type (FIG. 19D). Sod3 expression dropped below the detectable limit in wild type cells in high ambient copper (FIG. 19D) as expected, as did that of the pho84−/− null mutant. Hence, Sod3 expression is diminished in standard culture conditions in cells lacking Pho84, but can be induced in conditions of high abundance of its metal co-factor manganese.

Expression of SODs varies with metal availability in C. albicans. Since in S. cerevisiae, Pho84 under certain conditions transports manganese in addition to Pi, it was questioned whether lack of PHO84 might result in a manganese-depleted state in C. albicans which could lead to Sod3 deprivation and consequent failure to adequately manage ROS. In fact, upon measuring the intracellular manganese and copper concentrations, it was found that the opposite is true: both metals' concentrations were increased in pho84−/− null cells growing in standard synthetic complete medium, compared to wild type cells (FIGS. 19E and 19F). Manganese concentration in pho84−/− null cells supplemented with copper or manganese in the medium was like that of the wild type, while copper was increased in these cells under the same conditions (FIGS. 19E and 19F). Hence, lack of metal co-factors for SODs does not account for decreased Sod3 expression, or for slightly decreased Sod1 activity, in pho84−/− null cells.

### Example 13: Overexpression of a TORC1 Activator Downstream of Pho84 Suppresses ROS Management Defects of Pho84−/− Null Cells

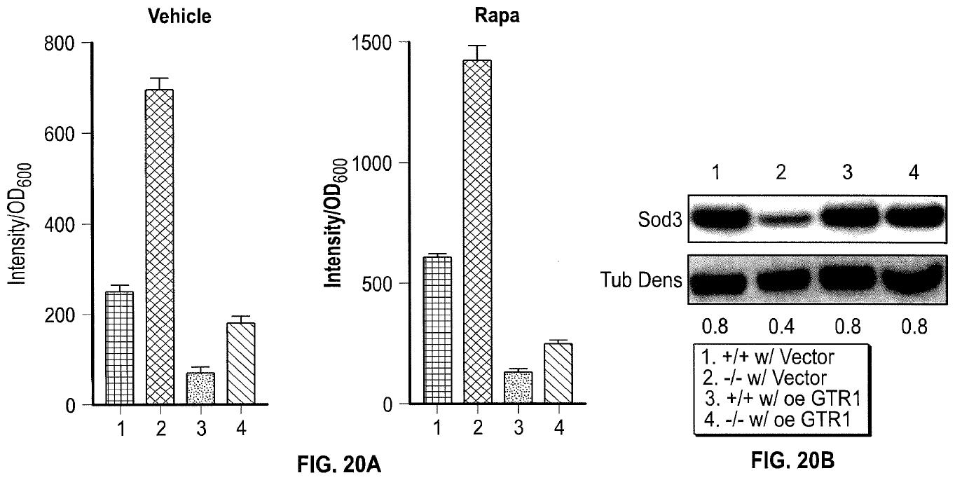

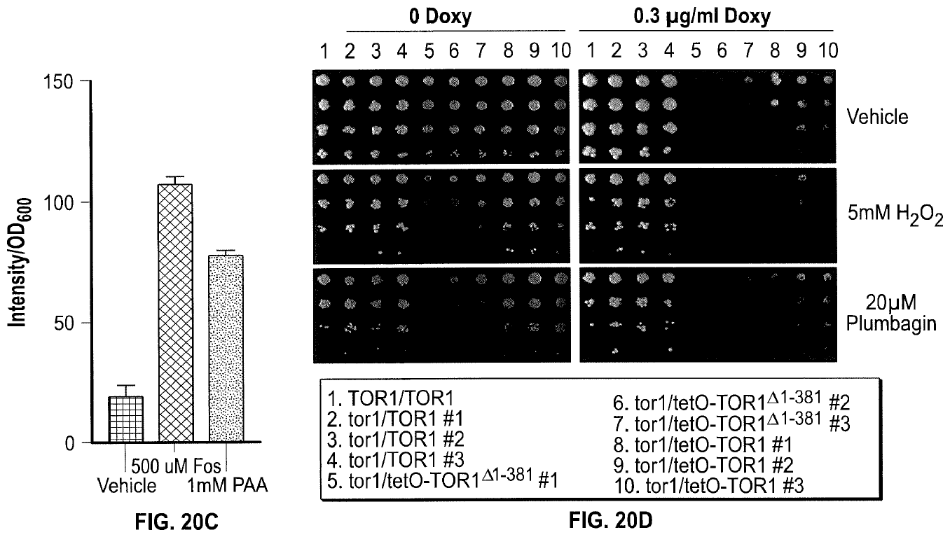

In S. cerevisiae, decreased TORC1 signaling increases mitochondrial ROS production. C. albicans cells lacking Pho84 exhibit decreased TORC1 signaling. This phenotype can be suppressed by overexpression of a component of the amino-acid signaling EGO complex, the small GTPase Gtr1, which may participate in transmitting a Pi signal to TORC1. It was questioned whether suppressing the TORC1 signaling defect of pho84−/− null cells by overexpressing GTR1 might improve ROS management of these cells. In the absence of exogenous oxidative stressors, GTR1 overexpression decreased the DCFDA detectable ROS in cells with active TORC1, as well as in cells experiencing TORC1 inhibition by exposure to a low concentration of rapamycin (FIG. 20A). Sod3 expression was increased in cells overexpressing GTR1 (FIG. 20B). Previously Applicant showed that inhibition of Pho84 with small molecules, phosphonoacetic acid (PAA) and phosphonoformic acid (foscarnet, Fos) which is an FDA-approved antiviral agent, leads to decreased TORC1 signaling. To test whether small-molecule Pho84 inhibition also leads to defective ROS management, wild type cells were exposed to these small molecules and measured their DCFDA detectable ROS. Exposure to Pho84 inhibitors increased the ROS content of wild type cells unexposed to external oxidative stressors, suggesting that the ROS detoxifying role of Pho84 can be targeted pharmacologically. In order to confirm that a ROS management defect could in fact be attributable to TORC1 perturbation, cells derived from 3 lineages of tor1/TOR1 heterozygotes, expressing the gene encoding the Tor1 kinase, and an N-terminally truncated hypomorphic Tor1Δ1-381, exclusively from the repressible tetO promoter, were subjected to oxidative stress during tetO induction and -repression. During TOR1 overexpression in the absence of doxycycline, cells expressing Tor1Δ1-381 were hypersensitive to oxidative stress. During TOR1 repression in the presence of doxycycline, cells containing both conditional TOR1 alleles were hypersensitive (FIG. 20C). These findings suggest that the role of Pho84 in oxidative stress management is connected to its role in activating TORC1.

### Example 14: Pho84 is Required for Virulence Toward In Vitro & Ex Vivo Host Cell Models & for Resistance to Neutrophil Killing

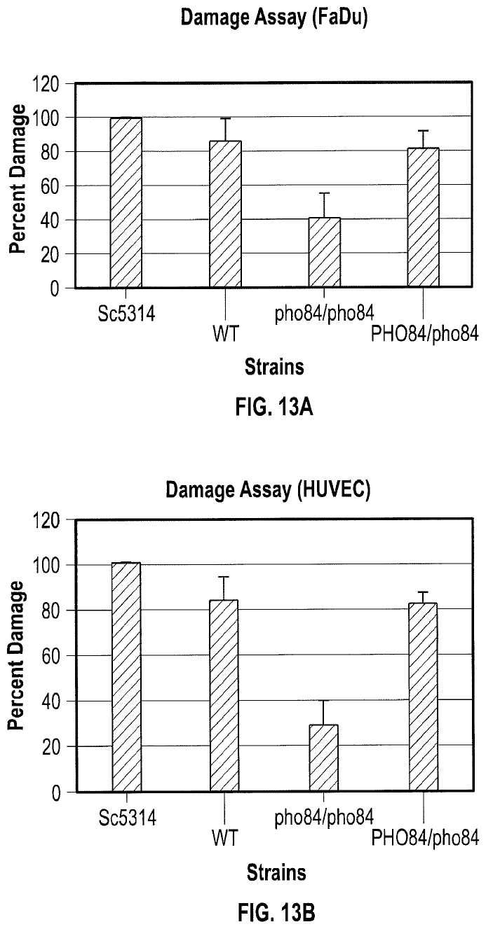

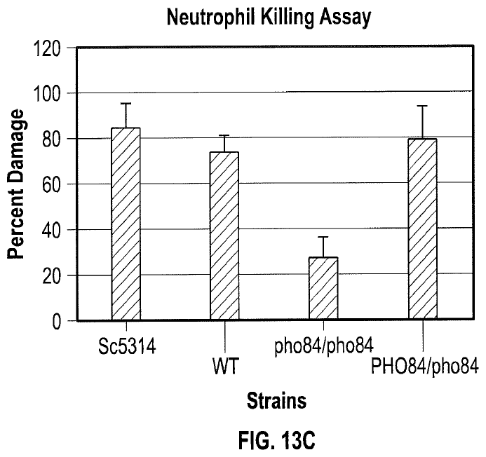

The extent of damage caused by C. albicans strains to human umbilical vein endothelial cells and the FaDu epithelial cell line was measured using a ⁵¹Cr release assay. Mammalian cells were grown in a 96-well tissue culture plate and incubated overnight with Na₂⁵¹CrO₄. The following day, endothelial were infected with 4×10⁴ C. albicans cells in RPMI, while FaDu cells were infected with 10⁵ organisms in the same medium. After a 3-h incubation, the upper 50% of medium was removed from each well and the amount of ⁵¹Cr in the aspirates and the well was determined by gamma counting. The data are presented in FIG. 13, panels A and B.

C. albicans strains were incubated with HL-60 derived neutrophil-like cultured cells for 90 minutes at 37° C. with 5% CO₂. Percent killing was calculated by dividing the number of CFU after co-culturing with HL-60 derived neutrophils by the number of CFU from C. albicans incubated with media without HL-60 derived neutrophils. HL-60 derived neutrophils were tested at a 20:1 phagocyte:fungus ratio in RPMI plus 10% pooled human serum. FIG. 13, panel C.

### Example 15: Pho84 is Required for Resistance to Platelet-Dependent Human Whole Blood Inhibition of C. albicans

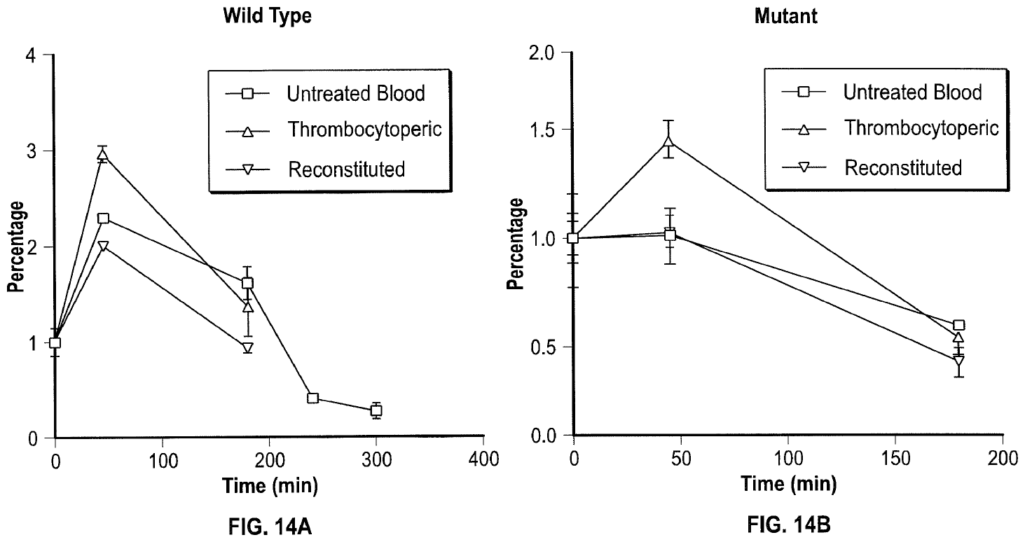

Human whole blood was depleted of platelets to a count of 50,000/ml and half of the blood aliquot was reconstituted to a platelet count of ˜200,000/ml. C. albicans PHO84/PHO84 wild type or pho84/pho84 null mutant cells from a washed overnight were inoculated to a density of 2×10³/ml and cfu counts were obtained by plating on YPD at the indicated time points. 2 biological replicates only to date. The data are presented in FIG. 14.

### Example 16: Neutrophil Extracellular Traps (NETs) Results in Rapid Damage to the Pho84−/− Mutant

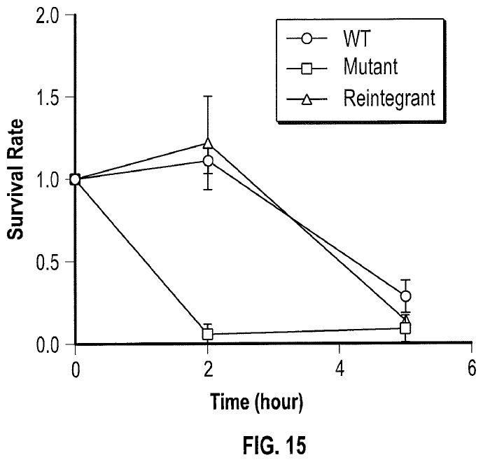

Cells from YPD overnight were washed with 0.9% NaCl and exposed to freshly isolated human neutrophils forming neutrophil extracellular traps (NETs), at a multiplicity of infection of 2. At 2 and 5 hours, neutrophils were lysed by exposure to 0.1% TritonX, cells were washed in water and plated for cfu counts. Data are presented in FIG. 15.

### Example 17: High Throughput Screen for Inhibitors of Pho84

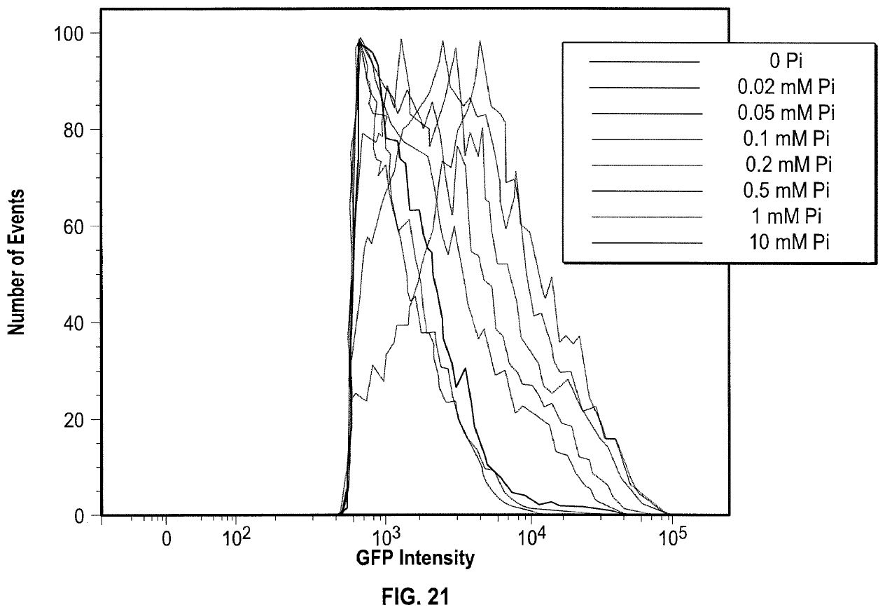

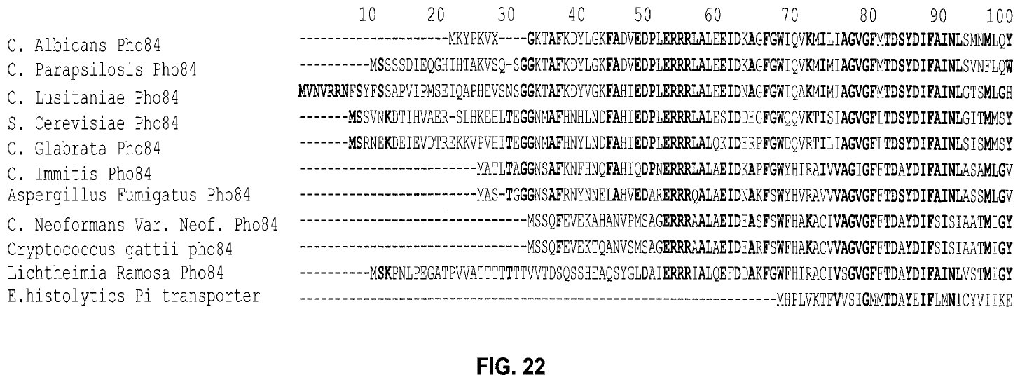

For high-throughput screening, a reporter strain, hemizygous for PHO84 and hence sensitized to Pho84 inhibitors, was generated, in which GFP is expressed from the PHO84 promoter. In cells transferred from PHO84-repressing (Pi rich) to -inducing (Pi poor) media, GFP is induced by ca. an order of magnitude per flow cytometry assay (FIG. 21). The advantage of screening with this reporter strain is its specificity to Pho84, because compounds that pleiotropically damage the cell will lead to reduced GFP synthesis and diminished fluorescence.

This reagent can also be used, together with pho84/PHO84 strains without GFP expression, as a chemical-genetic screenable sensitized mutant, because it expresses a single allele of PHO84 and hence is more sensitive to inhibitors than the wild type. By simple growth assays, such as those relying on optical density or absorption at 600 nm, or those relying on reduction of resazurin by metabolically active cells, these strains will exhibit more profound growth failure in low-phosphate media at 0.2 to 0.5 mM KH₂PO₄ than wild-type comparators.

Pho84 is highly conserved among fungal species, and a more distant homolog is even conserved in the pathogenic amoeba Entamoeba histolytica (FIG. 22) so that its inhibitors are predicted to be active against at least the following species which are pathogens and/or opportunistic pathogens of humans (under the heading of the bolded phylum name):

Ascomycota

Candida parapsilosis, C. glabrata, C. lusitaniae, C. krusei, C. dublinensis, C. kefyr, C. auris

Pneumocystis jirovecii

Aspergillus fumigatus, A. flavus, A. niger, A. terreus, A. nidulans

Fusarium solani, F. oxysporum, Fusarium verticillioidis and Fusarium moniliforme, Fusarium dimerum, Fusarium proliferatum, Fusarium chlamidosporum, Fusarium sacchari, Fusarium nygamai, Fusarium napiforme, Fusarium antophilum, and Fusarium vasinfectum

Pseudoallescheria boydii, Scedosporium apiospermum, S. prolificans

Alternaria alternata, Acremonium kiliense

Blastomyces dermatitidis

Histoplasma capsulatum

Coccidioides immitis, C. posadasii

Paracoccidioides brasiliensis

Trichophyton rubrum, T. tonsurans, T. schoenleinii, T. verrucosum, T. mentagrophytes, T. interdigitale, T. violaceum

Microsporum canis, M. audouinii, M. ferrugineum, M. cookei

Nannizzia fulva, N. gypsea, N. persicolor, N. nana

Arthroderma insingulare, A. uncinatum

Epidermophyton floccosum

Basidiomycota

Cryptococcus gattii, Cryptococcus neoformans

Trichosporon asahii

Mucorales

Mucor mucedo, M. circinelloides

Rhizopus oryzae

Cunninghamella bertholletiae

Lichtheimia ramosa

Rhizomucor pusillus

Saksenaea vasiformis

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of inhibiting Pho84 in a fungus comprising administering to the fungus an effective amount of a compound of formula 1: 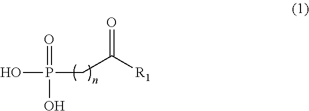
wherein n is 0 or 1 and R₁ is halogen, C₁-C₃ alkoxy, OH, H, or NH₂;
or a salt or solvate thereof, thereby inhibiting Pho84 in the fungus.

2. A method of inhibiting fungal growth comprising administering to a fungus an effective amount of the compound of formula 1 of claim 1: 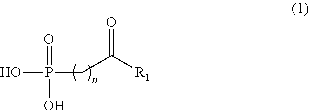
wherein n is 0 or 1 and R₁ is halogen, C₁-C₃ alkoxy, OH, H, or NH₂;
or a salt or solvate thereof, thereby inhibiting fungal growth.

3. A method of treating an infection caused by a fungus in a subject in need thereof comprising administering to the subject a therapeutically effective amount of the compound of formula 1 of claim 1: 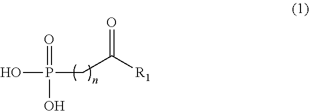
wherein n is 0 or 1 and R₁ is halogen, C₁-C₃ alkoxy, OH, H, or NH₂;
or a salt or solvate thereof, thereby treating the fungal infection in the subject.

4. The method of claim 1, wherein the compound is phosphonoformic acid or phosphonoacetic acid.

5. The method of claim 3, wherein the therapeutically effective amount of the compound of formula 1 is administered in a pharmaceutical composition comprising at least one pharmaceutically acceptable carrier.

6. The method of claim 1, wherein the fungus is from the phyla Zygomycete/Mucorales, Ascomycete, or Basidiomycete.

7. The method of claim 1, wherein the fungus is selected from the group consisting of: Candida parapsilosis, C. glabrata, C. lusitaniae, C. krusei, C. dublinensis, C. kefyr, C. auris, Pneumocystis jirovecii, Aspergillus fumigatus, A. flavus, A. niger, A. terreus, A. nidulans, Fusarium solani, F. oxysporum, Fusarium verticillioidis and Fusarium moniliforme, Fusarium dimerum, Fusarium proliferatum, Fusarium chlamidosporum, Fusarium sacchari, Fusarium nygamai, Fusarium napiforme, Fusarium antophilum, and Fusarium vasinfectum Pseudoallescheria boydii, Scedosporium apiospermum, S. prolificans, Alternaria alternata, Acremonium kiliense, Blastomyces dermatitidis, Histoplasma capsulatum, Coccidioides immitis, C. posadasii, Paracoccidioides brasiliensis, Trichophyton rubrum, T. tonsurans, T. schoenleinii, T. verrucosum, T. mentagrophytes, T. interdigitale, T. violaceum, Microsporum canis, M. audouinii, M. ferrugineum, M. cookie, Nannizzia fulva, N. gypsea, N. persicolor, N. nana Arthroderma insingulare, A. uncinatum, Epidermophyton floccosum, Cryptococcus Cryptococcus neoformans, Trichosporon asahii, Mucor mucedo, M. circinelloides Rhizopus oryzae, Cunninghamella bertholletiae, Lichtheimia ramose, Rhizomucor pusillus and Saksenaea vasiformis.

8. The method of claim 1, wherein the fungus is a yeast.

9. The method of claim 8, wherein the yeast is from the genera Candida or Cryptococcus.

10. The method of claim 8, wherein the yeast is Candida albicans or Saccharomyces cerevisiae.

11. The method of claim 1, further comprising administering at least one additional antifungal agent.

12. The method of claim 11, wherein the at least one additional antifungal agent is a polyene antifungal agent, an azole antifungal agent, an allylamine antifungal agent, or an echinocandin antifungal agent.

13. The method of claim 11, wherein the at least one additional antifungal agent is plumbagin, amphotericin B or micafungin.

14. A recombinant fungal cell comprising a polynucleotide encoding a reporter gene operably linked to a Pho84 promoter, wherein the fungal cell is hemizygous for Pho84.

15. An isolated polynucleotide encoding a reporter gene operably linked to a Pho84 promoter.

16. The isolated polynucleotide of claim 15, comprising the sequence of SEQ ID NO: 1.

17. An expression vector comprising the polynucleotide of claim 16 positioned for expression in a cell.

18. A cell comprising the expression vector of claim 17.

19. The cell of claim 18, wherein the cell is a fungal cell that is hemizygous for Pho84.

20. A recombinant fungal cell comprising polynucleotide of claim 16.

21. The recombinant fungal cell of claim 20, wherein the polynucleotide has at least 90% or 95% identity to SEQ ID NO: 1.

22. A method of identifying a compound that inhibits Pho84 comprising:
exposing the recombinant fungal cell of claim 14 to a putative inhibitor compound;
measuring the expression level of the reporter gene; and
comparing the expression level of the reporter gene to a predetermined reference level; thereby identifying the compound as a Pho84 inhibitor.

23-24. (canceled)

25. A composition comprising at least one Pho84+/− fungus cell and cell culture medium.

26-28. (canceled)

29. A method of identifying a compound that inhibits Pho84 comprising:
exposing at least one Pho84+/+ fungal cell and at least one Pho84+/− fungal cell of claim 25 to a putative inhibitor compound; and
comparing a growth level of the at least one Pho84+/+ fungal cell to a growth level of the at least one Pho84+/− fungal cell, thereby identifying the compound as a Pho84 inhibitor.

30-33. (canceled)

34. A kit for identifying a compound that inhibits Pho84, comprising:
one or more recombinant fungal cells comprising a polynucleotide encoding a reporter gene operably linked to a Pho84 promoter, wherein the fungal cell is hemizygous for Pho84;
a reagent for measuring the expression level of the reported gene;
and written instructions for identifying a compound that inhibits Pho84 accordance with the method of claim 22.

35. A kit for identifying a compound that inhibits Pho84, comprising:
one or more Pho84+/+ fungal cells and one or more Pho84+/− fungal cells;
a reagent for measuring the growth level of the fungal cells; and
written instructions for identifying a compound that inhibits Pho84 in accordance with the method of claim 29.


SEQUENCE LISTING (16 sequences)

SEQ ID NO: 1 - DNA, 1717 bp, Artificial Sequence
  Description of Artificial Sequence Synthetic polynucleotide
caaaaaatctaataattacaaaacttgtaaagcataaaactaaaaactttttattaataaaaagaaatatctcgtttgtatgttaaattgttgtacacatgggcaattacttggattggctaaacgaaataataaaaatgttgccattctagttttaaatactttatgattttagatgcattattacatttcgatttagtcatgtaaatgattagtgtaccatgtttggttggtaattgcctccccattcaagataactacactcatgtcttacccagacctccctatatgccactccaaactatccaacccttatagagatcacccttttaatcctccaattcatagattattaaccttgacatagctaccagatataaaatattcaaattttttttgttctcaacttcactctattctccattttttacagtttacaggaacattcccaaaagaagcaaaaaaaaataaaaattgtaagacttgaattacacaaaataaattatagtttttttttttgcactaaaattcaatttacacaaaatttaataatttctgaaaagtatcagtatcatcatcaccaagaaaacgcagccttcaaataaaaactcattttaattaaaactctcttatgaaattaaattcatccctaatagaaaggaaaggaaaaaaaaaaaaagaaatctcttgcatatctcgttatatattttgtatgaaataatataataaatatatacttcaaattagaatatataaatatttaaaacaattcccttaaattattgaaatttttatttttttataattcttcttcttcttttctttaaggactatttcaatatctcaattgttatttcactttattttccttcatatttacttattcattgggtttcctgcgtatatacataccctcccccctctttttgctatatcacatttacaactaacactatagtttttacaaaaagatggttgctgaaattcaarsctcacactaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 2 - protein, 238 aa, Artificial Sequence
  Description of Artificial Sequence Synthetic polypeptide
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - protein, 545 aa, Candida albicans
  MOD_RES (7)..(7): Any amino acid
MKYPKVXGKTAFKDYLGKFADVEDPLERRRLALEEIDKAGFGWTQVKMILIAGVGFMTDSYDIFAINLSMNMLQYVYWNGTIPDSTTTLIKVSTSVGTVIGQISFGSIADKVGRKKIYGLELIIMIFATLFQCTLGTSPAINFVAIFSTIRILMGIGIGGDYPLSSIISSEFSTTKWRGAIMAAVFSNQGLGQVFAGIVAMICVAGYKDDLIIANKGSECVGRCRKAADQMWRIIVGFGCVPGCIALYYRLTIAESPRYALDVDEHDDLEKVADAEAAIDVHAQEIAPPKASFKDFCSHFGQWRYGKILLGTAGSWFMLDVAYYGLGLNTTTILQTIGYAGQSNVYEKLYNSAAGNLILVCAGSLPGYWVSAATIDTVGRKPIQMGGFILLTIILCIMGFGYHKIGNHGLLGLFVIAQFFQNFGPNTTTFIVPGECFPTRYRSTAHGLSAAAGKVGAIIAQTCIGTLVNHGCSKENKNCFLPHVLEIFALFMLLGIGLTFLIPETARRTLEEISETCHGEVDTTKLGRDRFATQEEPYDSEEVKN

SEQ ID NO: 4 - protein, 587 aa, Saccharomyces cerevisiae
MSSVNKDTIHVAERSLHKEHLTEGGNMAFHNHLNDFAHIEDPLERRRLALESIDDEGFGWQQVKTISIAGVGFLTDSYDIFAINLGITMMSYVYWHGSMPGPSQTLLKVSTSVGTVIGQFGFGTLADIVGRKRIYGMELIIMIVCTILQTTVAHSPAINFVAVLTFYRIVMGIGIGGDYPLSSIITSEFATTKWRGAIMGAVFANQAWGQISGGIVALILVAAYKGELEYANSGAECDARCQKACDQMWRILIGLGTVLGLACLYFRLTIPESPRYQLDVNAKLELAAAAQEQDGEKKIHDTSDEDMAINGLERASTAVESLDNHPPKASFKDFCRHFGQWKYGKILLGTAGSWFTLDVAFYGLSLNSAVILQTIGYAGSKNVYKKLYDTAVGNLILICASGLPGYWCSVFTVDIIGRKPIQLAGFIILTALFCVIGFAYHKLGDHGLLALYVICQFFQNFGPNTTTFIVPGECFPTRYRSTAHGISAASGKVGAIIAQTALGTLIDHNCARDGKPTNCWLPHVMEIFALFMLLGIFTTLLIPETKRKTLEEINELYHDEIDPATLNFRNKNNDIESSSPSQLQHEA

SEQ ID NO: 5 - protein, 522 aa, Piriformospora indica
MLADLDHFGKNYKHDEEAQRNQKPWMLTWPQIKLVLLAGVGFFLDAYDLFIINQVAPMLAQVYFPKTGLPAQRQDLMKAAANIGCVVGQVMFGVLGDSFGRKFVYGKELILIIVATIFQMSAPSHWDGNRVLTWITICRVFLGIGIGGDYPMSATVVSDRANIHRRGTLLCFIFANQGWGSFVGSLVTIVTISGFKHRLKSGHTHDVDKAWRILIGLSLIPAFGTLYQRLTLPESRKFELTRDAASSSTVAIDKKDHDATHEVKDAPESEKSSPKVTPADAIDDDRHGVIASKKAHWQEFVAYFSTWNHFRNLLGSMLGWFLVDIAFYGINLNQSVVLAQIGFAGKTGDVYDKLFQLATGNIIVTALGFLPGYYFTLFLIDIVGRKKLQFMGFIMDSLFLAILAGEIDHIGKGPLLACFTFMQFFFNFGANTTTFIVAAELFPTRIRASAHGISAAAGKCGAILSSLVFNQLKAKIGTSAVLWIFFSTCILGFISTFLIDETMGVDPDEKDLEERRARGEIP

SEQ ID NO: 6 - protein, 265 aa, Candida albicans
  MOD_RES (7)..(7): Any amino acid
MKYPKVXGKTAFKDYLGKFADVEDPLERRRLALEEIDKAGFGWTQVKMILIAGVGFMTDSYDIFAINLSMNMLQYVYWNGTIPDSTTTLIKVSTSVGTVIGQISFGSIADKVGRKKIYGLELIIMIFATLFQCTLGTSPAINFVAIFSTIRILMGIGIGGDYPLSSIISSEFSTTKWRGAIMAAVFSNQGLGQVFAGIVAMICVAGYKDDLIIANKGSECVGRCRKAADQMWRIIVGFGCVPGCIALYYRLTIAESPRYALDVDE

SEQ ID NO: 7 - protein, 279 aa, Candida parapsilosis
MSSSSDIEQGHIHTAKVSQSGGKTAFKDYLGKFADVEDPLERRRLALEEIDKAGFGWTQVKMIMIAGVGFMTDSYDIFAINLSVNFLQWVYWNGTIPDSTNTLIKVATSVGTVIGQVGFGTVADIVGRKKIYGLELAIMIFATIAQVVLGESPAINFVAIFSSLRILMGIGIGGDYPLSSIISSEFSTTKWRGAIMAAVFSNQGLGQIFAGVVAMICVAGYKNDLIDYTKDTGCHGPCVKACDQMWRIVIGFGCVPGCIALYYRLTIAESPRYTLDMDE

SEQ ID NO: 8 - protein, 290 aa, Candida lusitaniae
MVNVRRNFSYFSSAPVIPMSEIQAPHEVSNSGGKTAFKDYVGKFAHIEDPLERRRLALEEIDNAGFGWTQAKMIMIAGVGFMTDSYDIFAINLGTSMLGHVYWGGTIPSSTQTLIKVSTSVGTVIGQVSFGMFADIVGRKKIYGLELIVMIFATVIQCTLGESAAISFPAIFSTMRILMGIGIGGDYPLSSIISSEFSTTKWRGAIMAAVFSNQGLGQLLAGIVALIAVSGYKDDLKYANTAKECIGSCIKANDQMWRILIGFGCVPGCIALYYRLTIAESPRYELDVQE

SEQ ID NO: 9 - protein, 285 aa, Saccharomyces cerevisiae
MSSVNKDTIHVAERSLHKEHLTEGGNMAFHNHLNDFAHIEDPLERRRLALESIDDEGFGWQQVKTISIAGVGFLTDSYDIFAINLGITMMSYVYWHGSMPGPSQTLLKVSTSVGTVIGQFGFGTLADIVGRKRIYGMELIIMIVCTILQTTVAHSPAINFVAVLTFYRIVMGIGIGGDYPLSSIITSEFATTKWRGAIMGAVFANQAWGQISGGIIALILVAAYKGELEYANSGAECDARCQKACDQMWRILIGLGTVLGLACLYFRLTIPESPRYQLDVNAKLE

SEQ ID NO: 10 - protein, 286 aa, Candida glabrata
MSRNEKDEIEVDTREKKVPVHITEGGNMAFHNYLNDFAHIEDPLERRRLALQKIDERPFGWDQVRTILIAGVGFLTDSYDIFAINLSISMMSYVFWQGNMPNSTQTLLKVSTSVGTVIGQLGFGTMADIVGRKKIYGLELIVMIATTILQTTIGRSPGINFPAVLTFYRIVMGIGIGGDYPLSSIITSEFATTKWRGAIMGAVFANQAWGQIAGGLVALIVIRAYKGDLIYANSGAECDYACQKACDQMWRILIGFGAVPGIIGLYFRLTIPESPRYALDVKEEVD

SEQ ID NO: 11 - protein, 274 aa, Coccidioides immitis
MATLTAGGNSAFKNFHNQFAHIQDPNERRRLALAEIDKAPFGWYHIRAIVVAGIGFFTDAYDIFAINLASAMLGVVFWLDAKDKPGKIPSSADTAIKVATSGGTVIGQVGFGWLADVVGRKKMYGLELMVIIFATLAQALSSDSPGVSIVGLLIFWRVIMGIGIGGDYPLSSIITSEFATTKWRGAMMGAVFAMQGIGQFAAALVALIVAAGFKESLKTAESESQCSGVCQVAVDKMWRVVIGFGAVPGCIALYYRLTIPETPRYTFDVQRDVV

SEQ ID NO: 12 - protein, 273 aa, Aspergillus fumigatus
MASTGGGNSAFRNYNNELAHVEDARERRRQALAEIDNAKFSWYHVRAVVVAGVGFFTDSYDIFAINLASSMLGVVFWQDAKSSPGKIPSSADTAIKVSTSGGTVIGQLFFGWLADRIRMYGIELMVIIMATLAQALSSDSRAGRKISIVGILIFWRVIMGIGIGGDYPLSSIITSEFATTKWRGAMMGSVFAMQGFGQFAAAIVALIVTAGFKESLETASSVGKCSGVCQLAVDKMWRVVIGFGAVPACIALYYRLTIPETPRYTFDVAHDIV

SEQ ID NO: 13 - protein, 256 aa, Cryptococcus neoformans
MSSQFEVEKAHANVPMSAGERRRAALAEIDEASFSWFHAKACIVAGVGFFTDAYDIFSISIAATMIGYVYHNGGSNTSNQDLGIKVAHSIGTFFGQLLFGYLADHIGRKRMYGVELIIIIIGTLGQAVAGHAPGVNIYGVLIMWRFIMGIGIGGDYPVSSVITSEFAARKIRGRMMAAVFSAQGWGNFTCAIVAVIVVAAYKDSIHSQPLDDLKSVDQAWRLIIGIGCVPAVIALYFRLTIPETPRYTMDVERNIK

SEQ ID NO: 14 - protein, 256 aa, Cryptococcus gattii
MSSQFEVEKTQANVSMSAGERRRAALAEIDEARFSWFHAKACVVAGVGFFTDAYDIFSISIAATMIGYVYHNGGSNTSNQDLGIKVAHSIGTFFGQLSFGYLADHIGRKRMYGVELMIIIVGTLGQAVAGHAPGVNIYGVLIMWRFIMGIGIGGDYPVSSVITSEFAARKIRGRMMAAVFSAQGWGNFACAIVAVIVVAAYKSSIKSQPLDDLKAVDQAWRLIIGIGCVPAVIALYFRLTIPETPRYTMDVERNIK

SEQ ID NO: 15 - protein, 276 aa, Lichtheimia ramosa
MSKPNLPEGATPVVATTTTTTTVVTDSQSSHEAQSYGLDAIERRRIALQEFDDAKFGWFHIRACIVSGVGFFTDAYDIFAINLVSTMIGYVYWGAEGNTTPHNVDTAIKVSMSCGTVVGQLLFGYLADHVGRKRMYGVELIIMIVGTIGQTLAGNGPAASFWAVITFWRVIVGIGIGGDYPLSSVITSEFATTKRRGAMMAAVFAMQGIGQVTAGIVGLIATAAYQNAIRADPTQLDYVWRIVIGLGAVPGVVALYYRLTIPETPRYTMDVEQKIE

SEQ ID NO: 16 - protein, 202 aa, Entamoeba histolytica
MHPLVKTFVVSIGMMTDAYEIFLMNICYVIIKEIYGETSYSSTLSSAVLVGMAIGQLLFGVLADTIGKKLMFITTLTILIIFSILQAFSFEVISGAGPYIILAVIRFILGLGIGGEYPLSSSIASEDAKPHNRGRKLVFIFSMQGIGNILAPLVVLILLYTPLKLDYVWRTALALGAVPCILTLYHRVIMDMPKKKKDHVTS